(19) United States
(12) Patent Application Publication
(10) Pub. No.: US 2008/0125427 A1
(43) Pub. Date: May 29, 2008

(54) Novel Inhibitors of Rho-Kinases

(75) Inventors: Clark A. Sehon, King of Prussia, PA (US); Dennis Lee, King Prussia, PA (US); Krista B. Goodman, King of Prussia, PA (US); Gren Z. Wang, King of Prussia, PA (US); Andrew Q. Viet, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 11/570,083

(22) PCT Filed: Jun. 17, 2005
(86) PCT No.: PCT/US05/21559

§ 371 Date: Aug. 17, 2007

Related U.S. Application Data
(60) Provisional application No. 60/580,423, filed on Jun. 17, 2004.

(51) Int. Cl. A61K 31/5377; C07D 403/12; A61K 31/506; A61P 9/04; A61P 9/10; A61P 9/12; A61P 25/28; C07D 413/14

(52) U.S. Cl. 514/234.5; 544/333; 514/256; 544/122

(57) ABSTRACT

Novel inhibitors of Rho-kinases are disclosed.

### FIELD OF THE INVENTION

The present invention relates to novel compounds and methods useful in the inhibition of rho-kinases.

### BACKGROUND OF THE INVENTION

An important large family of enzymes is the protein kinase enzyme family. Currently, there are about 500 different known protein kinases. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK), includes cyclic AMP- and cyclic GMP-dependent protein kinases, calcium- and phospholipid-dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also under progress to identify modulators of tyrosine kinases as well.

A major signal transduction systems utilized by cells is the RhoA-signalling pathways. RhoA is a small GTP binding protein that can be activated by several extracellular stimuli such as growth factor, hormones, mechanic stress, osmotic change as well as high concentration of metabolite like glucose. RhoA activation involves GTP binding, conformation alteration, post-translational modification (geranylgeranyllization and farnesylation) and activation of its intrinsic GTPase activity. Activated RhoA is capable of interacting with several effector proteins including Rho-Kinases (ROCK 1 and ROCK 2, referred to below as ‘ROCK’ or ‘ROCKs’) and transmit signals into cellular cytoplasm and nucleus.

ROCK1 and 2 constitute a family of kinases that can be activated by RhoA-GTP complex via physical association. Activated ROCKs phosphorylate a number of substrates and play important roles in pivotal cellular functions. The substrates for ROCKs include myosin binding subunit of myosin light chain phosphatase (MBS, also named MYPT1), adducin, moesin, myosin light chain (MLC), LIM kinase as well as transcription factor FHL. The phosphorylation of these substrates modulate the biological activity of the proteins and thus provide a means to alter cell's response to external stimuli. One well documented example is the participation of ROCK in smooth muscle contraction. Upon stimulation by phenylephrine, smooth muscle from blood vessels contracts. Studies have shown that phenylephrine stimulates b-adrenergic receptors and leads to the activation of RhoA. Activated RhoA in turn stimulates kinase activity of ROCK1 and which in turn phosphorylates MBS. Such phosphorylation inhibits the enzyme activity of myosin light chain phosphatase and increases the phosphorylation of myosin light chain itself by a calcium-dependent myosin light chain kinase (MLCK) and consequently increases the contractility of myosin-actin bundle, leading to smooth muscle contraction. This phenomenon is also sometimes called calcium sensitization. In addition to smooth muscle contraction, ROCKs have also been shown to be involved in cellular functions including apoptosis, cell migration, transcriptional activation, fibrosis, cytokinesis, inflammation and cell proliferation. Moreover, in neurons ROCK plays a critical role in the inhibition of axonal growth by myelin-associated inhibitory factors such as myelin-associated glycoprotein (MAG). ROCK-activity also mediates the collapse of growth cones in developing neurons. Both processes are thought to be mediated by ROCK-induced phosphorylation of substrates such as LIM kinase and myosin light chain phosphatase, resulting in increased contractility of the neuronal actin-myosin system.

The present inventors have discovered novel indazole compounds, which are inhibitors of ROCK activity and show interesting selectivity over other protein kinases. Such derivatives are useful in the treatment of disorders associated with inappropriate ROCK activity.

### SUMMARY OF THE INVENTION

This invention describes compounds and methods of using compounds for the inhibition of Rho-kinases. This invention also describes methods for the preparation of Rho-kinase inhibitory compounds as well as uses of said compounds in the manufacture of medicaments useful for the inhibition of Rho-kinases. Furthermore, this invention details the use of these compounds in compositions comprising at least one pharmaceutical excipient. In particular, this invention describes compounds of formula (I), wherein:

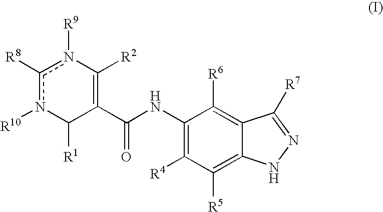

R⁸ is selected from the group consisting of aryl (optionally substituted with from 1-3 substituents selected from the group consisting of C₁₋₆ alkoxy, aryloxy, NR^{a}R^{b}, C₁₋₆ alkyl, SO₂C₁₋₆ alkyl, hydroxyl, halogen, cyano, nitro, C₁₋₆ thioalkyl, COOH, CO₂C₁₋₆ alkyl, and CONR^{a}R^{b}); NR^{a}R^{b}; heteroaryl (optionally substituted with 1-3 substituents selected from the group consisting of C₁₋₆ alkyl, NR^{a}R^{b}, halogen, cyano, nitro, C₁₋₆ alkoxy, aryloxy, COOH, CO₂C₁₋₆ alkyl, and CONR^{a}R^{b}, and C₁₋₆ thioalkyl); C₁₋₆ alkyl; C₁₋₆ alkylaryloxy; C₁₋₆ alkylaryl and C₃₋₇cycloalkyl;

### R⁹ is absent; hydrogen; or C₁₋₃ alkyl;

### R¹⁰ is absent; hydrogen; or C₁₋₃ alkyl;

R¹ is selected from the group consisting of aryl (optionally substituted with 1-3 substituents selected from the group consisting of halogen, cyano, nitro, C₁₋₆ alkyl, SO₂C₁₋₆alkyl, and SO₂NR^{c}R^{d}); C₁₋₆alkylaryl (wherein said alkyl portion is optionally substituted with thiophenyl); acylaryl; heteroaryl (optionally substituted with 1-3 substituents selected from the group consisting of halogen, C₁₋₃alkyl and aryl); C₁₋₆ alkyl; and C₃₋₇ cycloalkyl;

### R² is hydrogen or C₁₋₃ alkyl;

### R⁶ is hydrogen;

### R⁴ is hydrogen; C₁₋₃ alkyl; or halogen;

### R⁵ is hydrogen; C₁₋₃ alkyl; or halogen;

### R⁷ is hydrogen; halogen; or C₁₋₃ alkyl; and

R^{a} and R^{b} are each independently selected from the group consisting of hydrogen; C₁₋₆ alkyl; aryl; or taken together represent a ring containing from between 5 to 7 backbone atoms, wherein said ring is optionally substituted with from 1-3 substituents selected from C₁₋₃ alkyl; halogen; amino; or C₁₋₃ alkoxy; and one of the ring atoms optionally may be an additional heteroatom wherein said heteroatom is selected from the group consisting of N, O, or S);

### R^{c} and R^{d} are each independently selected from the group consisting of hydrogen and C₁₋₃ alkyl; and

pharmaceutically acceptable salts or solvates thereof.

### DETAILED DESCRIPTION OF THE INVENTION

The present invention includes compounds as described hereinbelow: The present invention thus provides compounds of the general formula (I), wherein:

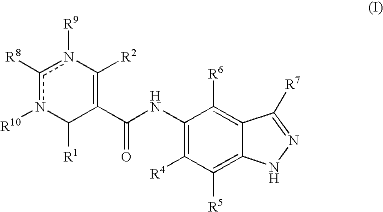

R⁸ is selected from the group consisting of aryl (optionally substituted with 1-3 substituents selected from the group consisting of C₁₋₆ alkoxy, aryloxy, NR^{a}R^{b}, C₁₋₆ alkyl, SO₂C₁₋₆ alkyl, hydroxyl, halogen, cyano, nitro, C₁₋₆ thioalkyl, COOH, CO₂C₁₋₆ alkyl, and CONR^{a}R^{b}); NR^{a}R^{b}; heteroaryl (optionally substituted with 1-3 substituents selected from the group consisting of C₁₋₆alkyl, NR^{a}R^{b}, halogen, cyano, nitro, C₁₋₆ alkoxy, aryloxy, COOH, CO₂C₁₋₆ alkyl, and CONR^{a}R^{b}, and C₁₋₆ thioalkyl); C₁₋₆ alkyl; C₁₋₆ alkylaryloxy; C₁₋₆alkylaryl and C₃₋₇cycloalkyl;

### R⁹ is absent; hydrogen; or C₁₋₃ alkyl;

### R¹⁰ is absent; hydrogen; or C₁₋₃ alkyl;

R¹ is selected from the group consisting of aryl (optionally substituted with 1-3 substituents selected from the group consisting of halogen, cyano, nitro, C₁₋₆ alkyl, SO₂C₁₋₆alkyl, and SO₂NR^{c}R^{d}); C₁₋₆alkylaryl (wherein said alkyl portion is optionally substituted with thiophenyl); acylaryl; heteroaryl (optionally substituted with 1-3 substituents selected from the group consisting of halogen, C₁₋₃alkyl and aryl); C₁₋₆ alkyl; and C₃₋₇cycloalkyl;

### R² is hydrogen or C₁₋₃ alkyl;

### R⁶ is hydrogen;

### R⁴ is hydrogen; C₁₋₃alkyl; or halogen;

### R⁵ is hydrogen; C₁₋₃ alkyl; or halogen;

### R⁷ is hydrogen; halogen; or C₁₋₃ alkyl; and

R^{a} and R^{b} are each independently selected from the group consisting of hydrogen; C₁₋₆ alkyl; aryl; or taken together represent a ring containing from between 5 to 7 backbone atoms, wherein said ring is optionally substituted with from 1-3 substituents selected from C₁₋₃ alkyl; halogen; amino; or C₁₋₃ alkoxy; and one of the ring atoms optionally may be an additional heteroatom wherein said heteroatom is selected from the group consisting of N, O, or S;

### R^{c} and R^{d} are each independently selected from the group consisting of hydrogen and C₁₋₃alkyl; and

pharmaceutically acceptable salts or solvates thereof.

In some embodiments, this invention describes a compound of formula (I) wherein R⁸ is optionally substituted pyridyl.

In some embodiments, this invention describes a compound of formula (I) wherein R⁸ is an optionally substituted 4-pyridyl.

In some embodiments, this invention describes a compound of formula (I) wherein R⁸ is optionally substituted phenyl.

In some embodiments, this invention describes a compound of formula (I) wherein R⁸ is phenyl optionally substituted at the 4-position.

In some embodiments, this invention describes a compound of formula (I) wherein R⁴ is hydrogen or fluorine.

In some embodiments, this invention describes a compound of formula (I) wherein R⁵ is hydrogen.

In some embodiments, this invention describes a compound of formula (I) wherein R⁷ is hydrogen.

In some embodiments, this invention describes a compound of formula (I) wherein R⁷ is methyl.

In some embodiments, this invention describes a compound of formula (I) wherein R⁷ is chlorine.

In some embodiments, this invention describes a compound of formula (I) wherein R¹ is substituted phenyl.

In some embodiments, this invention describes a compound of formula (I) wherein R⁹ and R¹⁰ are each independently absent or hydrogen.

In some embodiments, this invention describes a compound of formula (I) wherein said compound is 4-(4-fluoro-phenyl)-2-(4-methoxy-phenyl)-6-methyl-1,4-dihydro-pyrimidine-5-carboxylic acid (1H-indazol-5-yl)-amide; 6-(4-Chloro-2-fluorophenyl)-N-1H-indazol-5-yl-1,4-dimethyl-2-[4-(methyloxy)phenyl]-1,6-dihydro-5-pyrimidinecarboxamide; 4-(4-chloro-2-fluorophenyl)-N-1H-indazol-5-yl-6-methyl-2-[2-(methyloxy)-4-pyridinyl]-1,4-dihydro-5-pyrimidinecarboxamide; 2-amino-4-(4-fluorophenyl)-N-1H-indazol-5-yl-6-methyl-1,4-dihydro-5-pyrimidinecarboxamide; 4-(4-fluorophenyl)-N-1H-indazol-5-yl-2,6-dimethyl-1,4-dihydro-5-pyrimidinecarboxamide; 4-(4-fluorophenyl)-N-1H-indazol-5-yl-6-methyl-2-(phenylamino)-1,4-dihydro-5-pyrimidinecarboxamide; 2-cyclopropyl-4-(4-fluorophenyl)-N-1H-indazol-5-yl-6-methyl-1,4-dihydro-5-pyrimidinecarboxamide; 4-(4-fluorophenyl)-N-1H-indazol-5-yl-6-methyl-2-phenyl-1,4-dihydro-5-pyrimidinecarboxamide; N-1H-indazol-5-yl-2-(4-methoxyphenyl)-6-methyl-4-[4-(trifluoromethyl)phenyl]-1,4-dihydropyrimidine-5-carboxamide; 4-(4-fluorophenyl)-N-1H-indazol-5-yl-6-methyl-2-(3-methylphenyl)-1,4-dihydro-5-pyrimidinecarboxamide; 4-(4-fluorophenyl)-N-1H-indazol-5-yl-6-methyl-2-[4-(trifluoromethyl)phenyl]-1,4-dihydro-5-pyrimidinecarboxamide; 4-(4-fluorophenyl)-N-1H-indazol-5-yl-6-methyl-2-[3-(trifluoromethyl)phenyl]-1,4-dihydro-5-pyrimidinecarboxamide; 2-(4-chlorophenyl)-4-(4-fluorophenyl)-N-1H-indazol-5-yl-6-methyl-1,4-dihydro-5-pyrimidinecarboxamide; N-1H-indazol-5-yl-4-methyl-2-(3-methylphenyl)-6-[4-(trifluoromethyl)phenyl]-1,6-dihydro-5-pyrimidinecarboxamide; N-1H-indazol-5-yl-4-methyl-2,6-bis[4-(trifluoromethyl)phenyl]-1,6-dihydro-5-pyrimidinecarboxamide; N-1H-indazol-5-yl-4-methyl-2-[3-(trifluoromethyl)phenyl]-6-[4-(trifluoromethyl)phenyl]-1,6-dihydro-5-pyrimidinecarboxamide; 2-(4-chlorophenyl)-N-1H-indazol-5-yl-4-methyl-6-[4-(trifluoromethyl)phenyl]-1,6-dihydro-5-pyrimidinecarboxamide; 2-(3-fluoro-4-methylphenyl)-N-1H-indazol-5-yl-4-methyl-6-[4-(trifluoromethyl)phenyl]-1,6-dihydro-5-pyrimidinecarboxamide; 4-(4-fluorophenyl)-N-1H-indazol-5-yl-6-methyl-2-(4-methylphenyl)-1,4-dihydro-5-pyrimidinecarboxamide; 2-(3-fluoro-4-methylphenyl)-4-(4-fluorophenyl)-N-1H-indazol-5-yl-6-methyl-1,4-dihydro-5-pyrimidinecarboxamide; N-1H-indazol-5-yl-4-methyl-2-(4-methylphenyl)-6-[4-(trifluoromethyl)phenyl]-1,6-dihydro-5-pyrimidinecarboxamide; 2-(4-chlorophenyl)-N-1H-indazol-5-yl-4-methyl-6-(2-phenylethyl)-1,6-dihydro-5-pyrimidinecarboxamide; 2-(3-fluoro-4-methylphenyl)-N-1H-indazol-5-yl-4-methyl-6-(2-phenylethyl)-1,6-dihydro-5-pyrimidinecarboxamide; N-1H-indazol-5-yl-4-methyl-6-(2-phenylethyl)-2-[4-(trifluoromethyl)phenyl]-1,6-dihydro-5-pyrimidinecarboxamide; N-1H-indazol-5-yl-4-methyl-6-(2-phenylethyl)-2-[3-(trifluoromethyl)phenyl]-1,6-dihydro-5-pyrimidinecarboxamide; N-1H-indazol-5-yl-4-methyl-2-(3-methylphenyl)-6-(2-phenylethyl)-1,6-dihydro-5-pyrimidinecarboxamide; N-1H-indazol-5-yl-4-methyl-2-(4-methylphenyl)-6-(2-phenylethyl)-1,6-dihydro-5-pyrimidinecarboxamide; 2-[4-(aminocarbonyl)phenyl]-N-1H-indazol-5-yl-4-methyl-6-(2-phenylethyl)-1,6-dihydro-5-pyrimidinecarboxamide; 4-(4-fluorophenyl)-N-1H-indazol-5-yl-6-methyl-2-(2-pyridinyl)-1,4-dihydro-5-pyrimidinecarboxamide; 4-(4-fluorophenyl)-N-1H-indazol-5-yl-6-methyl-2-[6-(trifluoromethyl)-3-pyridinyl]-1,4-dihydro-5-pyrimidinecarboxamide; 4-(4-fluorophenyl)-N-1H-indazol-5-yl-6-methyl-2-(2-pyrazinyl)-1,4-dihydro-5-pyrimidinecarboxamide; 4-(4-fluorophenyl)-N-1H-indazol-5-yl-6-methyl-2-(2-thienyl)-1,4-dihydro-5-pyrimidinecarboxamide; 4-(4-fluorophenyl)-N-1H-indazol-5-yl-6-methyl-2-(3-pyridinyl)-1,4-dihydro-5-pyrimidinecarboxamide; 4-(4-fluorophenyl)-N-1H-indazol-5-yl-6-methyl-2-(2-methyl-1,3-thiazol-4-yl)-1,4-dihydro-5-pyrimidinecarboxamide; 2-(2,3-dihydro-1,4-benzodioxiN-2-yl)-4-(4-fluorophenyl)-N-1H-indazol-5-yl-6-methyl-1,4-dihydro-5-pyrimidinecarboxamide; 4-(4-fluorophenyl)-N-1H-indazol-5-yl-6-methyl-2-[(phenyloxy)methyl]-1,4-dihydro-5-pyrimidinecarboxamide; 4-(4-fluorophenyl)-N-1H-indazol-5-yl-6-methyl-2-(4-pyridinyl)-1,4-dihydro-5-pyrimidinecarboxamide; 4-(4-fluorophenyl)-N-1H-indazol-5-yl-6-methyl-2-(1-oxido-2-pyridinyl)-1,4-dihydro-5-pyrimidinecarboxamide; 2-(4-chlorophenyl)-N-1H-indazol-5-yl-4-methyl-6-(phenylcarbonyl)-1,6-dihydro-5-pyrimidinecarboxamide; N-1H-indazol-5-yl-4-methyl-6-phenyl-2-[4-(trifluoromethyl)phenyl]-1,6-dihydro-5-pyrimidinecarboxamide; N-1H-indazol-5-yl-4-methyl-2-[3-(methyloxy)phenyl]-6-[4-(trifluoromethyl)phenyl]-1,6-dihydro-5-pyrimidinecarboxamide; 4-[2-fluoro-4-(trifluoromethyl)phenyl]-N-1H-indazol-5-yl-6-methyl-2-[4-(trifluoromethyl)phenyl]-1,4-dihydro-5-pyrimidinecarboxamide; 4-(4-chloro-2-fluorophenyl)-N-1H-indazol-5-yl-6-methyl-2-[4-(trifluoromethyl)phenyl]-1,4-dihydro-5-pyrimidinecarboxamide; N-1H-indazol-5-yl-4-methyl-6-(5-phenyl-2-thienyl)-2-[4-(trifluoromethyl)phenyl]-1,6-dihydro-5-pyrimidinecarboxamide; N-1H-indazol-5-yl-4-methyl-2-(4-pyridinyl)-6-[4-(trifluoromethyl)phenyl]-1,6-dihydro-5-pyrimidinecarboxamide; N-1H-indazol-5-yl-4-methyl-6-(5-phenyl-2-thienyl)-2-(4-pyridinyl)-1,6-dihydro-5-pyrimidinecarboxamide; N-1H-indazol-5-yl-4,6-dimethyl-2-[4-(methyloxy)phenyl]-1,4-dihydro-5-pyrimidinecarboxamide; 4-(4-chloro-2-fluorophenyl)-N-1H-indazol-5-yl-6-methyl-2-[4-(methyloxy)phenyl]-1,4-dihydro-5-pyrimidinecarboxamide; 4-[2-fluoro-4-(trifluoromethyl)phenyl]-N-1H-indazol-5-yl-6-methyl-2-[4-(methyloxy)phenyl]-1,4-dihydro-5-pyrimidinecarboxamide; N-1H-indazol-5-yl-4-methyl-2-[4-(methyloxy)phenyl]-6-(5-phenyl-2-thienyl)-1,6-dihydro-5-pyrimidinecarboxamide; 4-(1-benzofuraN-2-yl)-N-1H-indazol-5-yl-6-methyl-2-[4-(methyloxy)phenyl]-1,4-dihydro-5-pyrimidinecarboxamide; N-1H-indazol-5-yl-4-methyl-6-[2-phenyl-2-(phenylthio)ethyl]-2-[4-(trifluoromethyl)phenyl]-1,6-dihydro-5-pyrimidinecarboxamide; 4-(1,3-benzodioxol-5-yl)-N-1H-indazol-5-yl-6-methyl-2-(4-pyridinyl)-1,4-dihydro-5-pyrimidinecarboxamide; 4-[2-fluoro-4-(trifluoromethyl)phenyl]-N-1H-indazol-5-yl-6-methyl-2-(4-pyridinyl)-1,4-dihydro-5-pyrimidinecarboxamide; 4-(4-chloro-2-fluorophenyl)-N-1H-indazol-5-yl-6-methyl-2-(4-pyridinyl)-1,4-dihydro-5-pyrimidinecarboxamide; N-1H-indazol-5-yl-4-methyl-2-[4-(methylthio)phenyl]-6-[4-(trifluoromethyl)phenyl]-1,6-dihydro-5-pyrimidinecarboxamide; 2-(2,3-dimethylphenyl)-N-1H-indazol-5-yl-4-methyl-6-[4-(trifluoromethyl)phenyl]-1,6-dihydro-5-pyrimidinecarboxamide; 2-(2,5-dimethylphenyl)-N-1H-indazol-5-yl-4-methyl-6-[4-(trifluoromethyl)phenyl]-1,6-dihydro-5-pyrimidinecarboxamide; 2-(4-chloro-3-methylphenyl)-N-1H-indazol-5-yl-4-methyl-6-[4-(trifluoromethyl)phenyl]-1,6-dihydro-5-pyrimidinecarboxamide; 2-[2-chloro-5-(trifluoromethyl)phenyl]-N-1H-indazol-5-yl-4-methyl-6-[4-(trifluoromethyl)phenyl]-1,6-dihydro-5-pyrimidinecarboxamide; 2-[3,4-bis(methyloxy)phenyl]-N-1H-indazol-5-yl-4-methyl-6-[4-(trifluoromethyl)phenyl]-1,6-dihydro-5-pyrimidinecarboxamide; N-1H-indazol-5-yl-4-methyl-2-[2-(methyloxy)phenyl]-6-[4-(trifluoromethyl)phenyl]-1,6-dihydro-5-pyrimidinecarboxamide; 2-[2,3-bis(methyloxy)phenyl]-N-1H-indazol-5-yl-4-methyl-6-[4-(trifluoromethyl)phenyl]-1,6-dihydro-5-pyrimidinecarboxamide; 2-(2-chlorophenyl)-N-1H-indazol-5-yl-4-methyl-6-[4-(trifluoromethyl)phenyl]-1,6-dihydro-5-pyrimidinecarboxamide; 2-(3-chlorophenyl)-N-1H-indazol-5-yl-4-methyl-6-[4-(trifluoromethyl)phenyl]-1,6-dihydro-5-pyrimidinecarboxamide; 4-(4-chloro-2-fluorophenyl)-2-(2-chloro-4-pyridinyl)-N-1H-indazol-5-yl-6-methyl-1,4-dihydro-5-pyrimidinecarboxamide; 2-(2-chloro-4-pyridinyl)-N-1H-indazol-5-yl-4-methyl-6-[4-(trifluoromethyl)phenyl]-1,6-dihydro-5-pyrimidinecarboxamide; 2-(2-chloro-4-pyridinyl)-4-[2-fluoro-4-(trifluoromethyl)phenyl]-N-1H-indazol-5-yl-6-methyl-1,4-dihydro-5-pyrimidinecarboxamide; 4-(4-chloro-2-fluorophenyl)-N-1H-indazol-5-yl-6-methyl-2-(4-morpholinyl)-1,4-dihydro-5-pyrimidinecarboxamide; 4-(4-chloro-2-fluorophenyl)-N-1H-indazol-5-yl-6-methyl-2-{[4-(methyloxy)phenyl]methyl}-1,4-dihydro-5-pyrimidinecarboxamide; 4-(4-chloro-2-fluorophenyl)-N-1H-indazol-5-yl-6-methyl-2-(2-thioxo-2,3-dihydro-1H-benzimidazol-5-yl)-1,4-dihydro-5-pyrimidinecarboxamide; 2-(2-chloro-4-pyridinyl)-N-1H-indazol-5-yl-4,6-dimethyl-1,4-dihydro-5-pyrimidinecarboxamide; 4-(4-chloro-2-fluorophenyl)-2-(2-chloro-4-pyridinyl)-N-(6-fluoro-1H-indazol-5-yl)-6-methyl-1,4-dihydro-5-pyrimidinecarboxamide; 4-(4-chloro-2-fluorophenyl)-N-1H-indazol-5-yl-6-methyl-2-[6-(methyloxy)-3-pyridinyl]-1,4-dihydro-5-pyrimidinecarboxamide; 4-(4-chloro-2-fluorophenyl)-2-(2,6-dichloro-4-pyridinyl)-N-1H-indazol-5-yl-6-methyl-1,4-dihydro-5-pyrimidinecarboxamide; N-1H-indazol-5-yl-4-methyl-2-[4-(methyloxy)phenyl]-6-phenyl-1,6-dihydro-5-pyrimidinecarboxamide; 4-(4-chloro-2-fluorophenyl)-2-{2-[(4-chlorophenyl)oxy]-4-pyridinyl}-N-1H-indazol-5-yl-6-methyl-1,4-dihydro-5-pyrimidinecarboxamide; 4-[4-(aminosulfonyl)phenyl]-N-1H-indazol-5-yl-6-methyl-2-[4-(methyloxy)phenyl]-1,4-dihydro-5-pyrimidinecarboxamide; N-1H-indazol-5-yl-4-methyl-2-[4-(methyloxy)phenyl]-6-[4-(methylsulfonyl)phenyl]-1,6-dihydro-5-pyrimidinecarboxamide; 4-(4-chloro-2-fluorophenyl)-2-[(4-chlorophenyl)methyl]-N-1H-indazol-5-yl-6-methyl-1,4-dihydro-5-pyrimidinecarboxamide; 4-(4-chloro-2-fluorophenyl)-N-1H-indazol-5-yl-6-methyl-2-(methylsulfonyl)-1,4-dihydro-5-pyrimidinecarboxamide; 2-(2-chloro-4-pyridinyl)-4-(4-fluorophenyl)-N-1H-indazol-5-yl-6-methyl-1,4-dihydro-5-pyrimidinecarboxamide; 4-(1,3-benzodioxol-5-yl)-2-(2-chloro-4-pyridinyl)-N-1H-indazol-5-yl-6-methyl-1,4-dihydro-5-pyrimidinecarboxamide; 2-(2-chloro-4-pyridinyl)-N-1H-indazol-5-yl-4-methyl-6-(2-methylpropyl)-1,6-dihydro-5-pyrimidinecarboxamide; 2-(2-chloro-4-pyridinyl)-4-(2,2-dimethylpropyl)-N-1H-indazol-5-yl-6-methyl-1,4-dihydro-5-pyrimidinecarboxamide; 2-(2-chloro-4-pyridinyl)-4-cyclopropyl-N-1H-indazol-5-yl-6-methyl-1,4-dihydro-5-pyrimidinecarboxamide; 2-(2-chloro-4-pyridinyl)-N-1H-indazol-5-yl-4-methyl-6-(phenylmethyl)-1,6-dihydro-5-pyrimidinecarboxamide; 2-(2-chloro-4-pyridinyl)-N-1H-indazol-5-yl-4-methyl-6-(2-phenylethyl)-1,6-dihydro-5-pyrimidinecarboxamide; 2-(2-chloro-4-pyridinyl)-4-ethyl-N-1H-indazol-5-yl-6-methyl-1,4-dihydro-5-pyrimidinecarboxamide; 2-(2-chloro-4-pyridinyl)-N-1H-indazol-5-yl-4-methyl-6-propyl-1,6-dihydro-5-pyrimidinecarboxamide; 2-(2-chloro-4-pyridinyl)-4-(1,1-dimethylethyl)-N-1H-indazol-5-yl-6-methyl-1,4-dihydro-5-pyrimidinecarboxamide; 4-(1,3-benzodioxol-5-yl)-N-(6-fluoro-1H-indazol-5-yl)-6-methyl-2-[2-(methyloxy)-4-pyridinyl]-1,4-dihydro-5-pyrimidinecarboxamide; N-(6-fluoro-1H-indazol-5-yl)-4-methyl-2-[2-(methyloxy)-4-pyridinyl]-6-[4-(trifluoromethyl)phenyl]-1,6-dihydro-5-pyrimidinecarboxamide; N-(6-fluoro-1H-indazol-5-yl)-4-[2-fluoro-4-(trifluoromethyl)phenyl]-6-methyl-2-[2-(methyloxy)-4-pyridinyl]-1,4-dihydro-5-pyrimidinecarboxamide; 4-(4-chloro-2-fluorophenyl)-N-(6-fluoro-1H-indazol-5-yl)-6-methyl-2-[2-(methyloxy)-4-pyridinyl]-1,4-dihydro-5-pyrimidinecarboxamide; N-(6-fluoro-1H-indazol-5-yl)-4-(4-fluorophenyl)-6-methyl-2-[2-(methyloxy)-4-pyridinyl]-1,4-dihydro-5-pyrimidinecarboxamide; N-(6-fluoro-1H-indazol-5-yl)-4-(4-fluorophenyl)-6-methyl-2-[6-(methyloxy)-3-pyridinyl]-1,4-dihydro-5-pyrimidinecarboxamide; 4-(4-chloro-2-fluorophenyl)-2-(2-chloro-4-pyridinyl)-N-(6-fluoro-1H-indazol-5-yl)-1,6-dimethyl-1,4-dihydro-5-pyrimidinecarboxamide; 4-(1,3-benzodioxol-5-yl)-N-(6-fluoro-1H-indazol-5-yl)-6-methyl-2-[6-(methyloxy)-3-pyridinyl]-1,4-dihydro-5-pyrimidinecarboxamide; N-(6-fluoro-1H-indazol-5-yl)-4-methyl-2-[6-(methyloxy)-3-pyridinyl]-6-[4-(trifluoromethyl)phenyl]-1,6-dihydro-5-pyrimidinecarboxamide; N-(6-fluoro-1H-indazol-5-yl)-4-[2-fluoro-4-(trifluoromethyl)phenyl]-6-methyl-2-[6-(methyloxy)-3-pyridinyl]-1,4-dihydro-5-pyrimidinecarboxamide; 4-(4-chloro-2-fluorophenyl)-N-(6-fluoro-1H-indazol-5-yl)-6-methyl-2-[6-(methyloxy)-3-pyridinyl]-1,4-dihydro-5-pyrimidinecarboxamide; 4-(4-chlorophenyl)-2-(2-chloro-4-pyridinyl)-N-(6-fluoro-1H-indazol-5-yl)-6-methyl-1,4-dihydro-5-pyrimidinecarboxamide; 4-(4-chlorophenyl)-N-(6-fluoro-1H-indazol-5-yl)-6-methyl-2-[2-(methyloxy)-4-pyridinyl]-1,4-dihydro-5-pyrimidinecarboxamide; 4-(4-chlorophenyl)-N-(6-fluoro-1H-indazol-5-yl)-6-methyl-2-[6-(methyloxy)-3-pyridinyl]-1,4-dihydro-5-pyrimidinecarboxamide; N-(3-chloro-6-fluoro-1H-indazol-5-yl)-4-(4-chloro-2-fluorophenyl)-2-(2-chloro-4-pyridinyl)-6-methyl-1,4-dihydro-5-pyrimidinecarboxamide; N-(3-chloro-6-fluoro-1H-indazol-5-yl)-4-(4-chloro-2-fluorophenyl)-6-methyl-2-[2-(methyloxy)-4-pyridinyl]-1,4-dihydro-5-pyrimidinecarboxamide; 4-(4-chloro-2-fluorophenyl)-N-1H-indazol-5-yl-2-[4-(methyloxy)phenyl]-6-(trifluoromethyl)-1,4-dihydro-5-pyrimidinecarboxamide; 2-amino-4-(4-chlorophenyl)-N-(6-fluoro-1H-indazol-5-yl)-6-methyl-1,4-dihydro-5-pyrimidinecarboxamide; 4-(4-chlorophenyl)-N-(6-fluoro-1H-indazol-5-yl)-2,6-dimethyl-1,4-dihydro-5-pyrimidinecarboxamide; 4-(4-chlorophenyl)-2-[2-(ethyloxy)-4-pyridinyl]-N-(6-fluoro-1H-indazol-5-yl)-6-methyl-1,4-dihydro-5-pyrimidinecarboxamide; 2-[2-(ethyloxy)-4-pyridinyl]-N-(6-fluoro-1H-indazol-5-yl)-4-[2-fluoro-4-(trifluoromethyl)phenyl]-6-methyl-1,4-dihydro-5-pyrimidinecarboxamide; 4-(4-chloro-2-fluorophenyl)-2-[2-(ethyloxy)-4-pyridinyl]-N-(6-fluoro-1H-indazol-5-yl)-6-methyl-1,4-dihydro-5-pyrimidinecarboxamide; 4-(4-chlorophenyl)-N-(6-fluoro-1H-indazol-5-yl)-6-methyl-2-(2-methyl-4-pyridinyl)-1,4-dihydro-5-pyrimidinecarboxamide; N-(6-fluoro-1H-indazol-5-yl)-4-[2-fluoro-4-(trifluoromethyl)phenyl]-6-methyl-2-(2-methyl-4-pyridinyl)-1,4-dihydro-5-pyrimidinecarboxamide; 4-(4-chloro-2-fluorophenyl)-N-(6-fluoro-1H-indazol-5-yl)-6-methyl-2-(2-methyl-4-pyridinyl)-1,4-dihydro-5-pyrimidinecarboxamide; 4-(4-chlorophenyl)-N-(6-fluoro-1H-indazol-5-yl)-6-methyl-2′-(methylthio)-1,4-dihydro-2,4′-bipyrimidine-5-carboxamide; 4-(4-chlorophenyl)-2-(2-chloro-4-pyridinyl)-6-methyl-N-(3-methyl-1H-indazol-5-yl)-1,4-dihydro-5-pyrimidinecarboxamide; 4-(4-chloro-2-fluorophenyl)-2-(2-chloro-4-pyridinyl)-6-methyl-N-(3-methyl-1H-indazol-5-yl)-1,4-dihydro-5-pyrimidinecarboxamide; 2-(2-chloro-4-pyridinyl)-4-[2-fluoro-4-(trifluoromethyl)phenyl]-6-methyl-N-(3-methyl-1H-indazol-5-yl)-1,4-dihydro-5-pyrimidinecarboxamide; N-(3-chloro-1H-indazol-5-yl)-4-(4-chlorophenyl)-2-(2-chloro-4-pyridinyl)-6-methyl-1,4-dihydro-5-pyrimidinecarboxamide; 4-(4-chloro-2-fluorophenyl)-N-(3-chloro-1H-indazol-5-yl)-2-(2-chloro-4-pyridinyl)-6-methyl-1,4-dihydro-5-pyrimidinecarboxamide, or a pharmaceutically acceptable salt or solvate thereof.

In some embodiments, this invention describes a composition containing a compound of formula (I) and a pharmaceutically acceptable excipient.

In some embodiments, this invention provides a use of a compound of formula (I) in the manufacture of a medicament for in the treatment of hypertension, alzheimer's disease, chronic and congestive heart failure and ischemic angina.

In some embodiments, this invention provides a method of treating a disease associated with excess Rho-kinase activity comprising the administration of a compound of formula (I) to a mammal in need thereof.

In some embodiments, this invention describes a method of treating hypertension, alzheimer's disease, chronic and congestive heart failure and ischemic angina comprising the administration of a compound of formula (I) to a mammal in need thereof.

In some embodiments, the methods of treatment of this invention are applied to a human in need thereof.

In certain aspects, this invention describes a process for the preparation of a compound of formula (I) comprising:
a) the coupling of a compound of formula (II) with an indazole of formula (III)

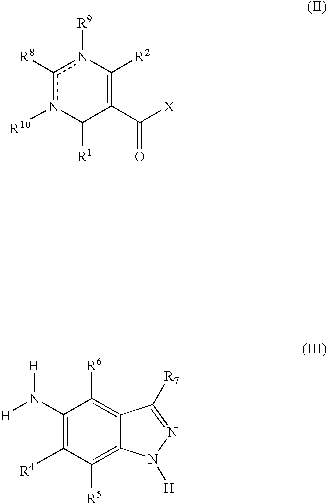

wherein R¹, R², R⁴, R⁵, R⁶, R⁷, R⁸, R⁹ and R¹⁰ are each as defined for formula (I), or are groups readily convertible thereto, and
x is OH, halogen, OC(═O)—C₁₋₆alkyl, or imidazole.

In yet another aspect, this invention describes a process for the preparation of a compound of formula (I), comprising the reaction between compounds of formula (IV), (V) and (VI) in the presence of a base:

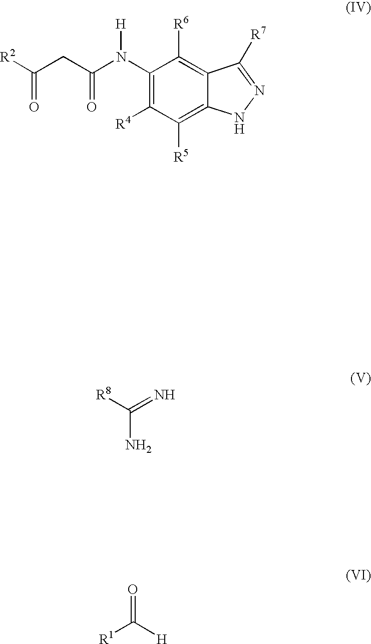

wherein R¹, R², R⁴, R⁵, R⁶, R⁷, and R⁸ are each as defined for formula (I), or are groups readily convertible thereto.

Wherein the R⁹ and R¹⁰ are defined as being, inter alia, “absent”, the said term “absent” means that the nitrogen atom does not bear the substituent. This would be the case where the nitrogen atom was present as an amidine-type moiety. This is not to say that where the nitrogen atom is present as an amidine moiety that it must be unsubstituted but only that it may be unsubstituted. Protonation or quaternization of the amidine nitrogen is still within the scope of this invention.

The term “alkyl” as a group or part of a group e.g. alkoxy, thioalkyl, alkylaryl, etc., contains the specified range of carbon atoms in the alkyl radical moiety (C₁₋₃ or C₁₋₆), wherein said alkyl radical moiety maybe straight chain or branched. For example, a C₁₋₃ alkyl substituent may include any of the following radicals: CH₃, CH₂CH₃, or CH(CH₃)₂. Said alkyl moiety may also be optionally substituted with halogen atoms. For example, CF₃, CH₂CF₃, CH(CF₃)₂, CHCICF₃, CH₂Br fit within the definition of C₁₋₃ alkyl radicals. Said alkyl may also be optionally substituted with one or more hydroxyl moieties, provided that two hydroxyl groups are not present on the same carbon atom. For example, C₁₋₃ alkyl may include CH₂OH, CH₂CH₂OH, C(OH)(CH₃)₂, CH(OH)—CH₂OH, and CH(CH₂OH)(CH₃)

The term “halogen” includes fluorine, chlorine, bromine or iodine.

The term “aryl” as a group or part of a group (e.g. aryloxy and aralkyl) refers to a phenyl, naphthyl, indane, or tetraline, and wherein said aryl group maybe optionally substituted with, if not specified otherwise, from 1-3 substituents selected from the group consisting of halogen, C₁₋₃ alkoxy, C₁₋₃ alkyl, cyano and nitro.

The term “heteroaryl” refers to a monocyclic, bicyclic or tricyclic ring system, containing from between 5 and 14 backbone atoms, and wherein at least one, but no more than 5 of the backbone atoms maybe a heteroatom. The heteroatoms are selected from the group consisting of N, O, or S, wherein said heteroaryl group maybe optionally substituted with, if not specified otherwise, from 1-3 substituents selected from the group consisting of halogens, C₁₋₃ alkoxy, C₁₋₃ alkyl, cyano and nitro. At least one of the rings in the system must be aromatic, but wherein a polycyclic compound is concerned, only one of the rings need be an aromatic ring. Also for purposes of this definition, one or more of the heteroatoms maybe located in a non-aromatic ring of a polycyclic heteroaromatic ring system. Furthermore, wherein the heteroatom(s) are located only in a non-aromatic ring of a polyaromatic, heteroaryl system, such systems will still be considered as “heteroaryl” for the purposes of the present application. Where the heteroaryl substituent includes an S atom in its backbone, said S-atom maybe present as S, SO, or SO₂, and where a heteroaryl backbone contains an N-atom, said N-atom may optionally be present as an N-oxide. Where the heteroaryl referred to herein contains at least one non-aromatic ring which then up to two of the carbon atoms in the heteroaryl ring system maybe present as —C(═O)— or —C(═S)—. Some non-limiting examples of heteroaryl ring systems include methylenedioxybenzene, ethylenedioxybenzene, pyridine, pyrimidine, quinoline, dihydroquinoline, 1,3-dihydro-2H-benzimidazole-2-thione, thiophene, thiazole, indole and benzofuran.

The compounds of formula (I) form salts with inorganic and organic acids and the invention includes such salts formed with physiologically acceptable inorganic and organic acids.

Inhibitors of ROCKs have been suggested for use in the treatments of a variety of diseases. They include cardiovascular diseases such as hypertension, chronic and congestive heart failure, ischemic angina, cardiac hypertrophy and fibrosis, restenosis, chronic renal failure and atherosclerosis. In addition, because of its muscle relaxing properties, it is also suitable for asthma, male erectile dysfunctions, female sexual dysfunction and over-active bladder syndrome. ROCK inhibitors have been shown to possess anti-inflammatory properties. Thus they can be used as treatment for neuroinflammatory diseases such as stroke, multiple sclerosis, Alzheimer's disease, Parkinson's disease, amyotrophic lateral sclerosis and inflammatory pain, as well as other inflammatory diseases such as rheumatoid arthritis, irritable bowel syndrome, inflammatory bowel disease, and Crohn's diseases. In addition, based on their neurite outgrowth inducing effects, ROCK inhibitors could be useful drugs for neuronal regeneration, inducing new axonal growth and axonal rewiring across lesions within the CNS. ROCK inhibitors are therefore likely to be useful for regenerative (recovery) treatment of CNS disorders such as spinal cord injury, acute neuronal injury (stroke, traumatic brain injury), Parkinsons disease, Alzheimers disease and other neurodegenerative disorders. Since ROCK inhibitors reduce cell proliferation and cell migration, they could be useful in treating cancer and tumor metastasis. Further more, there is evidence suggesting that ROCK inhibitors suppress cytoskeletal rearrangement upon virus invasion, thus they also have potential therapeutic value in anti-viral and anti-bacterial applications. ROCK inhibitors are also useful for the treatment of insulin resistance and diabetes.

Preferably ROCK inhibitors are useful for the treatment of hypertension, chronic and congestive heart failure, ischemic angina, asthma, alzheimer's disease, male erectile dysfunction, female sexual dysfunction, stroke, inflammatory bowel diseases, spinal cord injury, glaucoma and tumor metastasis.

More preferably ROCK inhibitors are useful for the treatment of hypertension, chronic and congestive heart failure, alzheimer and ischemic angina.

It is to be understood that for purposes of this invention, the term “treatment” includes the prevention or prophylaxis of a disease state.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula (I) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of formula (I) are included within the scope of the compounds of formula (I). In particular, it should be appreciated that the amidinine portion of the dihydro-pyrimidines of formula (I) may exist as one, or a mixture of tautomers and that although a structure might be named by assigning a specific tautomer to the formula, this does not imply that the compound consists of that tautomer but instead maybe a mixture of tautomers, or primarily or solely another tautomer.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a compound of formula (I), as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of compounds of the formula (I) and salts, solvates and physiological functional derivatives thereof, and one or more pharmaceutically acceptable carriers, diluents, or excipients. The compounds of the formula (I) and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I), and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the human or other animal, the precise condition requiring treatment and its severity, the nature of the formulation, and the route of administration, and will ultimately be at the discretion of the attendant physician or veterinarian. However, an effective amount of a compound of formula (I) for the treatment of neoplastic growth, for example colon or breast carcinoma, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula (I) per se. It is envisaged that similar dosages would be appropriate for treatment of the other conditions referred to above.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1999) Protecting Groups in Organic Synthesis, 3^{rd} edition, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I).

Compounds with the general structure 10 can be prepared according to the procedure described in Scheme 1. Treatment of an appropriately substituted nitrile 1 with Me₃Al/NH₄Cl provides substituted phenylamidines 3. Compounds of general structure 5 may be synthesized from compounds of general structure 3 by treatment with the appropriately substituted aldehyde 4, β-ketoester 2, and KOAc in DMSO. If desired, compounds of the general structure 5 can be further derivatized by treatment with the appropriate base such as NaH and an appropriate electrophile. Conversion of the esters 5 or 6 to a carboxylic acid 7 can be accomplished by several methods. This transformation is dependent upon the type of ester used, and can be accomplished with a variety of conditions for each type of ester, examples of which can be found in the literature, specifically “Protective Groups on Organic Synthesis” by Greene and Wuts. Acids of general structure 7 can be coupled with aminoindazoles of general formula 8 to afford substituted indazole amides 10, employing methods know to those skilled in the art (e.g. EDC, DMAP). Alternatively, compounds of general structure 10 can be accessed directly by reaction of ketoamides 9 with the appropriately substituted amidine 3 and aldehyde 4.

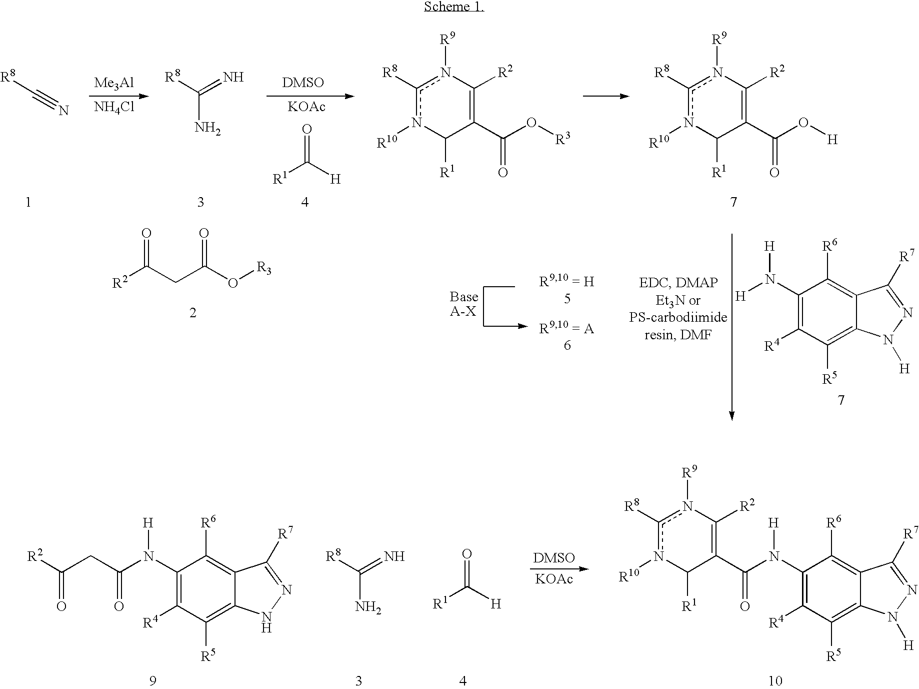

Examples of suitable compounds according to the invention include those listed below and found in Examples 1-2. Examples of this invention may also be prepared wherein the one or more of the substitutions of the starting material is a group that is readily converted to any one or more of R¹, R², R⁴, R⁵, R⁶, R⁷, and R⁸.

The following examples are intended to be illustrative only and not limiting in any way.
4-(4-chloro-2-fluorophenyl)-N-1H-indazol-5-yl-6-methyl-2-[2-(methyloxy)-4-pyridinyl]-1,4-dihydro-5-pyrimidinecarboxamide;
2-amino-4-(4-fluorophenyl)-N-1H-indazol-5-yl-6-methyl-1,4-dihydro-5-pyrimidinecarboxamide;
4-(4-fluorophenyl)-N-1H-indazol-5-yl-2,6-dimethyl-1,4-dihydro-5-pyrimidinecarboxamide;
4-(4-fluorophenyl)-N-1H-indazol-5-yl-6-methyl-2-(phenylamino)-1,4-dihydro-5-pyrimidinecarboxamide;
2-cyclopropyl-4-(4-fluorophenyl)-N-1H-indazol-5-yl-6-methyl-1,4-dihydro-5-pyrimidinecarboxamide;
4-(4-fluorophenyl)-N-1H-indazol-5-yl-6-methyl-2-phenyl-1,4-dihydro-5-pyrimidinecarboxamide;
N-1H-indazol-5-yl-2-(4-methoxyphenyl)-6-methyl-4-[4-(trifluoromethyl)phenyl]-1,4-dihydropyrimidine-5-carboxamide;
4-(4-fluorophenyl)-N-1H-indazol-5-yl-6-methyl-2-(3-methylphenyl)-1,4-dihydro-5-pyrimidinecarboxamide;
4-(4-fluorophenyl)-N-1H-indazol-5-yl-6-methyl-2-[4-(trifluoromethyl)phenyl]-1,4-dihydro-5-pyrimidinecarboxamide;
4-(4-fluorophenyl)-N-1H-indazol-5-yl-6-methyl-2-[3-(trifluoromethyl)phenyl]-1,4-dihydro-5-pyrimidinecarboxamide;
2-(4-chlorophenyl)-4-(4-fluorophenyl)-N-1H-indazol-5-yl-6-methyl-1,4-dihydro-5-pyrimidinecarboxamide;
N-1H-indazol-5-yl-4-methyl-2-(3-methylphenyl)-6-[4-(trifluoromethyl)phenyl]-1,6-dihydro-5-pyrimidinecarboxamide;
N-1H-indazol-5-yl-4-methyl-2,6-bis[4-(trifluoromethyl)phenyl]-1,6-dihydro-5-pyrimidinecarboxamide;
N-1H-indazol-5-yl-4-methyl-2-[3-(trifluoromethyl)phenyl]-6-[4-(trifluoromethyl)phenyl]-1,6-dihydro-5-pyrimidinecarboxamide;
2-(4-chlorophenyl)-N-1H-indazol-5-yl-4-methyl-6-[4-(trifluoromethyl)phenyl]-1,6-dihydro-5-pyrimidinecarboxamide;
2-(3-fluoro-4-methylphenyl)-N-1H-indazol-5-yl-4-methyl-6-[4-(trifluoromethyl)phenyl]-1,6-dihydro-5-pyrimidinecarboxamide;
4-(4-fluorophenyl)-N-1H-indazol-5-yl-6-methyl-2-(4-methylphenyl)-1,4-dihydro-5-pyrimidinecarboxamide;
2-(3-fluoro-4-methylphenyl)-4-(4-fluorophenyl)-N-1H-indazol-5-yl-6-methyl-1,4-dihydro-5-pyrimidinecarboxamide;
N-1H-indazol-5-yl-4-methyl-2-(4-methylphenyl)-6-[4-(trifluoromethyl)phenyl]-1,6-dihydro-5-pyrimidinecarboxamide;
2-(4-chlorophenyl)-N-1H-indazol-5-yl-4-methyl-6-(2-phenylethyl)-1,6-dihydro-5-pyrimidinecarboxamide;
2-(3-fluoro-4-methylphenyl)-N-1H-indazol-5-yl-4-methyl-6-(2-phenylethyl)-1,6-dihydro-5-pyrimidinecarboxamide;
N-1H-indazol-5-yl-4-methyl-6-(2-phenylethyl)-2-[4-(trifluoromethyl)phenyl]-1,6-dihydro-5-pyrimidinecarboxamide;
N-1H-indazol-5-yl-4-methyl-6-(2-phenylethyl)-2-[3-(trifluoromethyl)phenyl]-1,6-dihydro-5-pyrimidinecarboxamide;
N-1H-indazol-5-yl-4-methyl-2-(3-methylphenyl)-6-(2-phenylethyl)-1,6-dihydro-5-pyrimidinecarboxamide;
N-1H-indazol-5-yl-4-methyl-2-(4-methylphenyl)-6-(2-phenylethyl)-1,6-dihydro-5-pyrimidinecarboxamide;
2-[4-(aminocarbonyl)phenyl]-N-1H-indazol-5-yl-4-methyl-6-(2-phenylethyl)-1,6-dihydro-5-pyrimidinecarboxamide;
4-(4-fluorophenyl)-N-1H-indazol-5-yl-6-methyl-2-(2-pyridinyl)-1,4-dihydro-5-pyrimidinecarboxamide;
4-(4-fluorophenyl)-N-1H-indazol-5-yl-6-methyl-2-[6-(trifluoromethyl)-3-pyridinyl]-1,4-dihydro-5-pyrimidinecarboxamide;
4-(4-fluorophenyl)-N-1H-indazol-5-yl-6-methyl-2-(2-pyrazinyl)-1,4-dihydro-5-pyrimidinecarboxamide;
4-(4-fluorophenyl)-N-1H-indazol-5-yl-6-methyl-2-(2-thienyl)-1,4-dihydro-5-pyrimidinecarboxamide;
4-(4-fluorophenyl)-N-1H-indazol-5-yl-6-methyl-2-(3-pyridinyl)-1,4-dihydro-5-pyrimidinecarboxamide;
4-(4-fluorophenyl)-N-1H-indazol-5-yl-6-methyl-2-(2-methyl-1,3-thiazol-4-yl)-1,4-dihydro-5-pyrimidinecarboxamide;
2-(2,3-dihydro-1,4-benzodioxiN-2-yl)-4-(4-fluorophenyl)-N-1H-indazol-5-yl-6-methyl-1,4-dihydro-5-pyrimidinecarboxamide;
4-(4-fluorophenyl)-N-1H-indazol-5-yl-6-methyl-2-[(phenyloxy)methyl]-1,4-dihydro-5-pyrimidinecarboxamide;
4-(4-fluorophenyl)-N-1H-indazol-5-yl-6-methyl-2-(4-pyridinyl)-1,4-dihydro-5-pyrimidinecarboxamide;
4-(4-fluorophenyl)-N-1H-indazol-5-yl-6-methyl-2-(1-oxido-2-pyridinyl)-1,4-dihydro-5-pyrimidinecarboxamide;
2-(4-chlorophenyl)-N-1H-indazol-5-yl-4-methyl-6-(phenylcarbonyl)-1,6-dihydro-5-pyrimidinecarboxamide;
N-1H-indazol-5-yl-4-methyl-6-phenyl-2-[4-(trifluoromethyl)phenyl]-1,6-dihydro-5-pyrimidinecarboxamide;
N-1H-indazol-5-yl-4-methyl-2-[3-(methyloxy)phenyl]-6-[4-(trifluoromethyl)phenyl]-1,6-dihydro-5-pyrimidinecarboxamide;
4-[2-fluoro-4-(trifluoromethyl)phenyl]-N-1H-indazol-5-yl-6-methyl-2-[4-(trifluoromethyl)phenyl]-1,4-dihydro-5-pyrimidinecarboxamide;
4-(4-chloro-2-fluorophenyl)-N-1H-indazol-5-yl-6-methyl-2-[4-(trifluoromethyl)phenyl]-1,4-dihydro-5-pyrimidinecarboxamide;
N-1H-indazol-5-yl-4-methyl-6-(5-phenyl-2-thienyl)-2-[4-(trifluoromethyl)phenyl]-1,6-dihydro-5-pyrimidinecarboxamide;
N-1H-indazol-5-yl-4-methyl-2-(4-pyridinyl)-6-[4-(trifluoromethyl)phenyl]-1,6-dihydro-5-pyrimidinecarboxamide;
N-1H-indazol-5-yl-4-methyl-6-(5-phenyl-2-thienyl)-2-(4-pyridinyl)-1,6-dihydro-5-pyrimidinecarboxamide;
N-1H-indazol-5-yl-4,6-dimethyl-2-[4-(methyloxy)phenyl]-1,4-dihydro-5-pyrimidinecarboxamide;
4-(4-chloro-2-fluorophenyl)-N-1H-indazol-5-yl-6-methyl-2-[4-(methyloxy)phenyl]-1,4-dihydro-5-pyrimidinecarboxamide;
4-[2-fluoro-4-(trifluoromethyl)phenyl]-N-1H-indazol-5-yl-6-methyl-2-[4-(methyloxy)phenyl]-1,4-dihydro-5-pyrimidinecarboxamide;
N-1H-indazol-5-yl-4-methyl-2-[4-(methyloxy)phenyl]-6-(5-phenyl-2-thienyl)-1,6-dihydro-5-pyrimidinecarboxamide;
4-(1-benzofuraN-2-yl)-N-1H-indazol-5-yl-6-methyl-2-[4-(methyloxy)phenyl]-1,4-dihydro-5-pyrimidinecarboxamide;
N-1H-indazol-5-yl-4-methyl-6-[2-phenyl-2-(phenylthio)ethyl]-2-[4-(trifluoromethyl)phenyl]-1,6-dihydro-5-pyrimidinecarboxamide;
4-(1,3-benzodioxol-5-yl)-N-1H-indazol-5-yl-6-methyl-2-(4-pyridinyl)-1,4-dihydro-5-pyrimidinecarboxamide;
4-[2-fluoro-4-(trifluoromethyl)phenyl]-N-1H-indazol-5-yl-6-methyl-2-(4-pyridinyl)-1,4-dihydro-5-pyrimidinecarboxamide;
4-(4-chloro-2-fluorophenyl)-N-1H-indazol-5-yl-6-methyl-2-(4-pyridinyl)-1,4-dihydro-5-pyrimidinecarboxamide;
N-1H-indazol-5-yl-4-methyl-2-[4-(methylthio)phenyl]-6-[4-(trifluoromethyl)phenyl]-1,6-dihydro-5-pyrimidinecarboxamide;
2-(2,3-dimethylphenyl)-N-1H-indazol-5-yl-4-methyl-6-[4-(trifluoromethyl)phenyl]-1,6-dihydro-5-pyrimidinecarboxamide;
2-(2,5-dimethylphenyl)-N-1H-indazol-5-yl-4-methyl-6-[4-(trifluoromethyl)phenyl]-1,6-dihydro-5-pyrimidinecarboxamide;
2-(4-chloro-3-methylphenyl)-N-1H-indazol-5-yl-4-methyl-6-[4-(trifluoromethyl)phenyl]-1,6-dihydro-5-pyrimidinecarboxamide;
2-[2-chloro-5-(trifluoromethyl)phenyl]-N-1H-indazol-5-yl-4-methyl-6-[4-(trifluoromethyl)phenyl]-1,6-dihydro-5-pyrimidinecarboxamide;
2-[3,4-bis(methyloxy)phenyl]-N-1H-indazol-5-yl-4-methyl-6-[4-(trifluoromethyl)phenyl]-1,6-dihydro-5-pyrimidinecarboxamide;
N-1H-indazol-5-yl-4-methyl-2-[2-(methyloxy)phenyl]-6-[4-(trifluoromethyl)phenyl]-1,6-dihydro-5-pyrimidinecarboxamide;
2-[2,3-bis(methyloxy)phenyl]-N-1H-indazol-5-yl-4-methyl-6-[4-(trifluoromethyl)phenyl]-1,6-dihydro-5-pyrimidinecarboxamide;
2-(2-chlorophenyl)-N-1H-indazol-5-yl-4-methyl-6-[4-(trifluoromethyl)phenyl]-1,6-dihydro-5-pyrimidinecarboxamide;
2-(3-chlorophenyl)-N-1H-indazol-5-yl-4-methyl-6-[4-(trifluoromethyl)phenyl]-1,6-dihydro-5-pyrimidinecarboxamide;
4-(4-chloro-2-fluorophenyl)-2-(2-chloro-4-pyridinyl)-N-1H-indazol-5-yl-6-methyl-1,4-dihydro-5-pyrimidinecarboxamide;
2-(2-chloro-4-pyridinyl)-N-1H-indazol-5-yl-4-methyl-6-[4-(trifluoromethyl)phenyl]-1,6-dihydro-5-pyrimidinecarboxamide;
2-(2-chloro-4-pyridinyl)-4-[2-fluoro-4-(trifluoromethyl)phenyl]-N-1H-indazol-5-yl-6-methyl-1,4-dihydro-5-pyrimidinecarboxamide;
4-(4-chloro-2-fluorophenyl)-N-1H-indazol-5-yl-6-methyl-2-(4-morpholinyl)-1,4-dihydro-5-pyrimidinecarboxamide;
4-(4-chloro-2-fluorophenyl)-N-1H-indazol-5-yl-6-methyl-2-{[4-(methyloxy)phenyl]methyl}-1,4-dihydro-5-pyrimidinecarboxamide;
4-(4-chloro-2-fluorophenyl)-N-1H-indazol-5-yl-6-methyl-2-(2-thioxo-2,3-dihydro-1H-benzimidazol-5-yl)-1,4-dihydro-5-pyrimidinecarboxamide;
2-(2-chloro-4-pyridinyl)-N-1H-indazol-5-yl-4,6-dimethyl-1,4-dihydro-5-pyrimidinecarboxamide;
4-(4-chloro-2-fluorophenyl)-2-(2-chloro-4-pyridinyl)-N-(6-fluoro-1H-indazol-5-yl)-6-methyl-1,4-dihydro-5-pyrimidinecarboxamide;
4-(4-chloro-2-fluorophenyl)-N-1H-indazol-5-yl-6-methyl-2-[6-(methyloxy)-3-pyridinyl]-1,4-dihydro-5-pyrimidinecarboxamide;
4-(4-chloro-2-fluorophenyl)-2-(2,6-dichloro-4-pyridinyl)-N-1H-indazol-5-yl-6-methyl-1,4-dihydro-5-pyrimidinecarboxamide;
N-1H-indazol-5-yl-4-methyl-2-[4-(methyloxy)phenyl]-6-phenyl-1,6-dihydro-5-pyrimidinecarboxamide;
4-(4-chloro-2-fluorophenyl)-2-{2-[(4-chlorophenyl)oxy]-4-pyridinyl}-N-1H-indazol-5-yl-6-methyl-1,4-dihydro-5-pyrimidinecarboxamide;
4-[4-(aminosulfonyl)phenyl]-N-1H-indazol-5-yl-6-methyl-2-[4-(methyloxy)phenyl]-1,4-dihydro-5-pyrimidinecarboxamide;
N-1H-indazol-5-yl-4-methyl-2-[4-(methyloxy)phenyl]-6-[4-(methylsulfonyl)phenyl]-1,6-dihydro-5-pyrimidinecarboxamide;
4-(4-chloro-2-fluorophenyl)-2-[(4-chlorophenyl)methyl]-N-1H-indazol-5-yl-6-methyl-1,4-dihydro-5-pyrimidinecarboxamide;
4-(4-chloro-2-fluorophenyl)-N-1H-indazol-5-yl-6-methyl-2-(methylsulfonyl)-1,4-dihydro-5-pyrimidinecarboxamide;
2-(2-chloro-4-pyridinyl)-4-(4-fluorophenyl)-N-1H-indazol-5-yl-6-methyl-1,4-dihydro-5-pyrimidinecarboxamide;
4-(1,3-benzodioxol-5-yl)-2-(2-chloro-4-pyridinyl)-N-1H-indazol-5-yl-6-methyl-1,4-dihydro-5-pyrimidinecarboxamide;
2-(2-chloro-4-pyridinyl)-N-1H-indazol-5-yl-4-methyl-6-(2-methylpropyl)-1,6-dihydro-5-pyrimidinecarboxamide;
2-(2-chloro-4-pyridinyl)-4-(2,2-dimethylpropyl)-N-1H-indazol-5-yl-6-methyl-1,4-dihydro-5-pyrimidinecarboxamide;
2-(2-chloro-4-pyridinyl)-4-cyclopropyl-N-1H-indazol-5-yl-6-methyl-1,4-dihydro-5-pyrimidinecarboxamide;
2-(2-chloro-4-pyridinyl)-N-1H-indazol-5-yl-4-methyl-6-(phenylmethyl)-1,6-dihydro-5-pyrimidinecarboxamide;
2-(2-chloro-4-pyridinyl)-N-1H-indazol-5-yl-4-methyl-6-(2-phenylethyl)-1,6-dihydro-5-pyrimidinecarboxamide;
2-(2-chloro-4-pyridinyl)-4-ethyl-N-1H-indazol-5-yl-6-methyl-1,4-dihydro-5-pyrimidinecarboxamide;
2-(2-chloro-4-pyridinyl)-N-1H-indazol-5-yl-4-methyl-6-propyl-1,6-dihydro-5-pyrimidinecarboxamide;
2-(2-chloro-4-pyridinyl)-4-(1,1-dimethylethyl)-N-1H-indazol-5-yl-6-methyl-1,4-dihydro-5-pyrimidinecarboxamide;
4-(1,3-benzodioxol-5-yl)-N-(6-fluoro-1H-indazol-5-yl)-6-methyl-2-[2-(methyloxy)-4-pyridinyl]-1,4-dihydro-5-pyrimidinecarboxamide;
N-(6-fluoro-1H-indazol-5-yl)-4-methyl-2-[2-(methyloxy)-4-pyridinyl]-6-[4-(trifluoromethyl)phenyl]-1,6-dihydro-5-pyrimidinecarboxamide;
N-(6-fluoro-1H-indazol-5-yl)-4-[2-fluoro-4-(trifluoromethyl)phenyl]-6-methyl-2-[2-(methyloxy)-4-pyridinyl]-1,4-dihydro-5-pyrimidinecarboxamide;
4-(4-chloro-2-fluorophenyl)-N-(6-fluoro-1H-indazol-5-yl)-6-methyl-2-[2-(methyloxy)-4-pyridinyl]-1,4-dihydro-5-pyrimidinecarboxamide;
N-(6-fluoro-1H-indazol-5-yl)-4-(4-fluorophenyl)-6-methyl-2-[2-(methyloxy)-4-pyridinyl]-1,4-dihydro-5-pyrimidinecarboxamide;
N-(6-fluoro-1H-indazol-5-yl)-4-(4-fluorophenyl)-6-methyl-2-[6-(methyloxy)-3-pyridinyl]-1,4-dihydro-5-pyrimidinecarboxamide;
4-(4-chloro-2-fluorophenyl)-2-(2-chloro-4-pyridinyl)-N-(6-fluoro-1H-indazol-5-yl)-1,6-dimethyl-1,4-dihydro-5-pyrimidinecarboxamide;
4-(1,3-benzodioxol-5-yl)-N-(6-fluoro-1H-indazol-5-yl)-6-methyl-2-[6-(methyloxy)-3-pyridinyl]-1,4-dihydro-5-pyrimidinecarboxamide;
N-(6-fluoro-1H-indazol-5-yl)-4-methyl-2-[6-(methyloxy)-3-pyridinyl]-6-[4-(trifluoromethyl)phenyl]-1,6-dihydro-5-pyrimidinecarboxamide;
N-(6-fluoro-1H-indazol-5-yl)-4-[2-fluoro-4-(trifluoromethyl)phenyl]-6-methyl-2-[6-(methyloxy)-3-pyridinyl]-1,4-dihydro-5-pyrimidinecarboxamide;
4-(4-chloro-2-fluorophenyl)-N-(6-fluoro-1H-indazol-5-yl)-6-methyl-2-[6-(methyloxy)-3-pyridinyl]-1,4-dihydro-5-pyrimidinecarboxamide;
4-(4-chlorophenyl)-2-(2-chloro-4-pyridinyl)-N-(6-fluoro-1H-indazol-5-yl)-6-methyl-1,4-dihydro-5-pyrimidinecarboxamide;
4-(4-chlorophenyl)-N-(6-fluoro-1H-indazol-5-yl)-6-methyl-2-[2-(methyloxy)-4-pyridinyl]-1,4-dihydro-5-pyrimidinecarboxamide;
4-(4-chlorophenyl)-N-(6-fluoro-1H-indazol-5-yl)-6-methyl-2-[6-(methyloxy)-3-pyridinyl]-1,4-dihydro-5-pyrimidinecarboxamide;
N-(3-chloro-6-fluoro-1H-indazol-5-yl)-4-(4-chloro-2-fluorophenyl)-2-(2-chloro-4-pyridinyl)-6-methyl-1,4-dihydro-5-pyrimidinecarboxamide;
N-(3-chloro-6-fluoro-1H-indazol-5-yl)-4-(4-chloro-2-fluorophenyl)-6-methyl-2-[2-(methyloxy)-4-pyridinyl]-1,4-dihydro-5-pyrimidinecarboxamide;
4-(4-chloro-2-fluorophenyl)-N-1H-indazol-5-yl-2-[4-(methyloxy)phenyl]-6-(trifluoromethyl)-1,4-dihydro-5-pyrimidinecarboxamide;
2-amino-4-(4-chlorophenyl)-N-(6-fluoro-1H-indazol-5-yl)-6-methyl-1,4-dihydro-5-pyrimidinecarboxamide;
4-(4-chlorophenyl)-N-(6-fluoro-1H-indazol-5-yl)-2,6-dim ethyl-1,4-dihydro-5-pyrimidinecarboxamide;
4-(4-chlorophenyl)-2-[2-(ethyloxy)-4-pyridinyl]-N-(6-fluoro-1H-indazol-5-yl)-6-methyl-1,4-dihydro-5-pyrimidinecarboxamide;
2-[2-(ethyloxy)-4-pyridinyl]-N-(6-fluoro-1H-indazol-5-yl)-4-[2-fluoro-4-(trifluoromethyl)phenyl]-6-methyl-1,4-dihydro-5-pyrimidinecarboxamide;
4-(4-chloro-2-fluorophenyl)-2-[2-(ethyloxy)-4-pyridinyl]-N-(6-fluoro-1H-indazol-5-yl)-6-methyl-1,4-dihydro-5-pyrimidinecarboxamide;
4-(4-chlorophenyl)-N-(6-fluoro-1H-indazol-5-yl)-6-methyl-2-(2-methyl-4-pyridinyl)-1,4-dihydro-5-pyrimidinecarboxamide;
N-(6-fluoro-1H-indazol-5-yl)-4-[2-fluoro-4-(trifluoromethyl)phenyl]-6-methyl-2-(2-methyl-4-pyridinyl)-1,4-dihydro-5-pyrimidinecarboxamide;
4-(4-chloro-2-fluorophenyl)-N-(6-fluoro-1H-indazol-5-yl)-6-methyl-2-(2-methyl-4-pyridinyl)-1,4-dihydro-5-pyrimidinecarboxamide;
4-(4-chlorophenyl)-N-(6-fluoro-1H-indazol-5-yl)-6-methyl-2′-(methylthio)-1,4-dihydro-2,4′-bipyrimidine-5-carboxamide;
4-(4-chlorophenyl)-2-(2-chloro-4-pyridinyl)-6-methyl-N-(3-methyl-1H-indazol-5-yl)-1,4-dihydro-5-pyrimidinecarboxamide;
4-(4-chloro-2-fluorophenyl)-2-(2-chloro-4-pyridinyl)-6-methyl-N-(3-methyl-1H-indazol-5-yl)-1,4-dihydro-5-pyrimidinecarboxamide;
2-(2-chloro-4-pyridinyl)-4-[2-fluoro-4-(trifluoromethyl)phenyl]-6-methyl-N-(3-methyl-1H-indazol-5-yl)-1,4-dihydro-5-pyrimidinecarboxamide;
N-(3-chloro-1H-indazol-5-yl)-4-(4-chlorophenyl)-2-(2-chloro-4-pyridinyl)-6-methyl-1,4-dihydro-5-pyrimidinecarboxamide; and
4-(4-chloro-2-fluorophenyl)-N-(3-chloro-1H-indazol-5-yl)-2-(2-chloro-4-pyridinyl)-6-methyl-1,4-dihydro-5-pyrimidinecarboxamide.

### EXAMPLES

The following examples are intended to be illustrative only and not limiting in any way:

### Example 1

### 4-(4-Fluorophenyl)-N-1H-indazol-6-yl-6-methyl-2-[4-(methyloxy)phenyl]-1,4-dihydro-5-pyrimidinecarboxamide

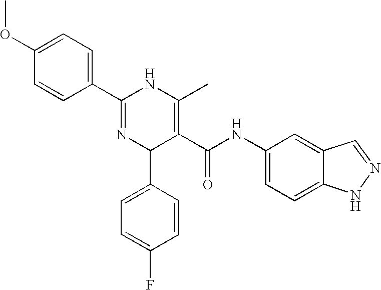

### Step 1. 4-(Methyloxy)benzenecarboximidamide

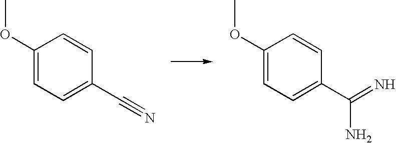

To a suspension of NH₄Cl (5.0 g, 93.5 mmol) in toluene (100 mL) with stirring was added Al(CH₃)₃ in toluene (47 mL, 2.0 M) at 0° C. over 10 min. The mixture was allowed to reach room temperature, stirred for 30 min, and a solution of 4-methoxybenzonitrile (12.6 g, 93.5 mmol) in toluene (20 mL) was injected. The mixture was heated at 80° C. for 16 hours, then cooled to room temperature, and poured into a slurry of silica gel (200 g) in CHCl₃ (500 mL). The mixture was stirred for 5 min and the silica gel was filtered. The filter cake was further washed with methanol (300 mL). Evaporation of the filtrate gave product (12.0 g, 85%) as a white solid.

### Step 2. 4-(4-Fluorophenyl)-N-1H-indazol-5-yl-6-methyl-2-[4-(methyloxy)phenyl]-1,4-dihydro-5-pyrimidinecarboxamide

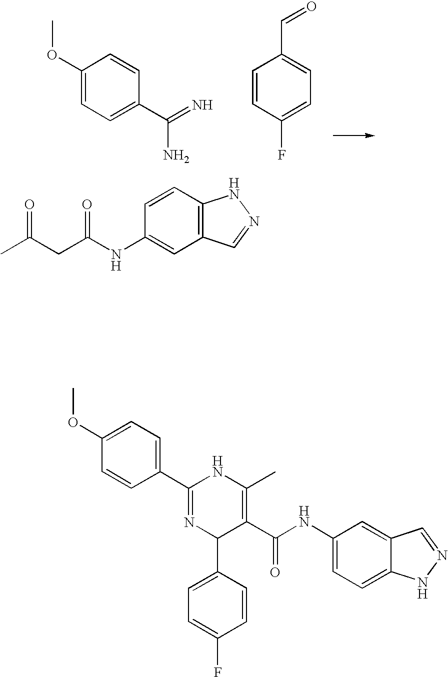

A mixture of 4-(methyloxy)benzenecarboximidamide (50 mg, 0.33 mmol), 4-fluorobenzaldehyde (35 mg, 0.28 mmol), N-1H-indazol-5-yl-3-oxobutanamide (60 mg, 0.28 mmol), and potassium acetate (55 mg, 0.56 mmol) was dissolved in DMSO (1 mL) with stirring, and heated at 100° C. for 3 hours. The reaction mixture was cooled to room temperature, poured into water (10 mL) to form a precipitate. The precipitate was filtered, washed with water, and purified by HPLC (RP, 25-75% CH₃CN/H₂O with 0.05 M NH₄HCO₃) to afford the desired product (41 mg, 32%) as a yellow solid. MS (ES+) m/z 456 [M+H]⁺.

### Example 2

### 6-(4-Chloro-2-fluorophenyl)-N-1H-indazol-5-yl-1,4-dimethyl-2-[4-(methyloxy)phenyl]-1,6-dihydro-5-pyrimidinecarboxamide

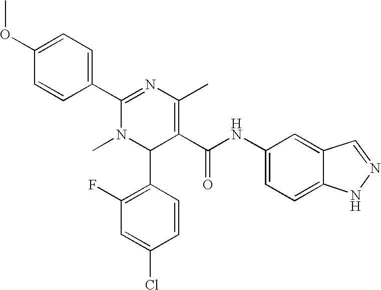

### Step 1. Methyl 4-(4-chloro-2-fluorophenyl)-6-methyl-2-[4-(methyloxy)phenyl]-1,4-dihydro-5-pyrimidinecarboxylate

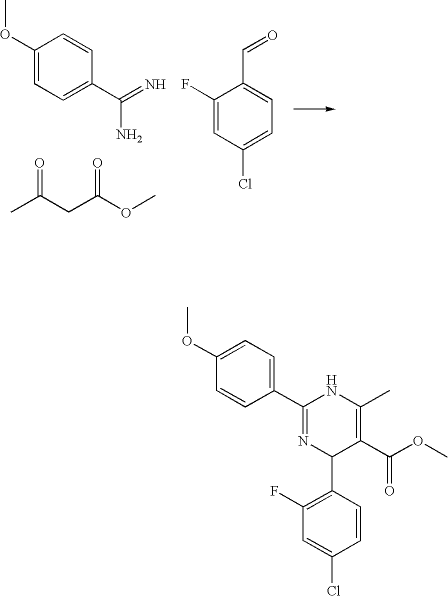

A mixture of 4-(methyloxy)benzenecarboximidamide (3.0 g, 20.0 mmol), 4-chloro-2-fluorobenzaldehyde (2.22 g, 14.3 mmol), methyl acetoacetate (2.0 g, 17.2 mmol), and potassium acetate (1.4 g, 14.3 mmol) was dissolved in DMSO (100 mL) with stirring, and heated at 80° C. for 16 hours. The reaction mixture was diluted with ethyl acetate, washed with brine (3×100 mL), and dried (MgSO₄). The solvent was removed via rotovap and the residue was purified by ISCO silica column to afford a greenish solid product (2.0 g, 36%). MS (ES+) m/z 390 [M+H]⁺.

### Step 2. Methyl 6-(4-chloro-2-fluorophenyl)-1,4-dimethyl-2-[4-(methyloxy)phenyl]-1,6-dihydro-5-pyrimidinecarboxylate

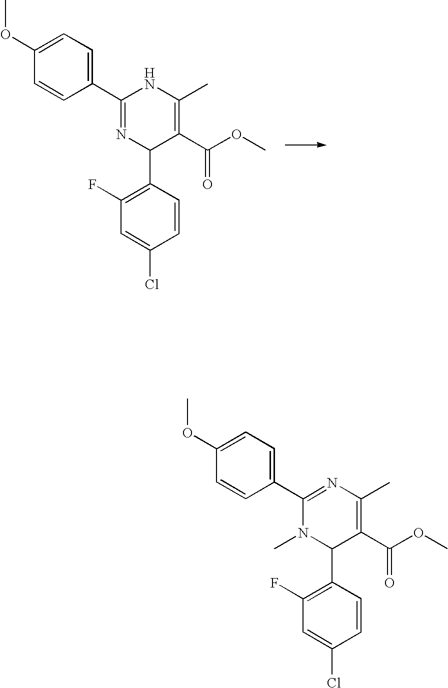

To a suspension of NaH (60%, 76 mg, 1.89 mmol) in THF (anhydrous, 1 mL) with stirring at 0° C. was added a solution of greenish solid (612 mg, 1.57 mmol) from above reaction in THF (anhydrous, 1 mL plus 1 mL rinse) dropwise. CH₃I (0.11 mL, 1.73 mmol) was added to the reaction mixture after 15 min. Cold bath was removed and the reaction mixture was stirred at room temperature for one hour. The reaction was quenched with water, extracted with ethyl acetate (3×5 mL). The extracts were combined, washed with water, brine, and dried (Na₂SO₄). The solvent was removed via rotovap to afford the title compound (619 mg, 98%) as oil. MS (ES+) m/z 403 [M+H]⁺.

### Step 3. 6-(4-Chloro-2-fluorophenyl)-1,4-dimethyl-2-[4-(methyloxy)phenyl]-1,6-dihydro-5-pyrimidinecarboxylic acid

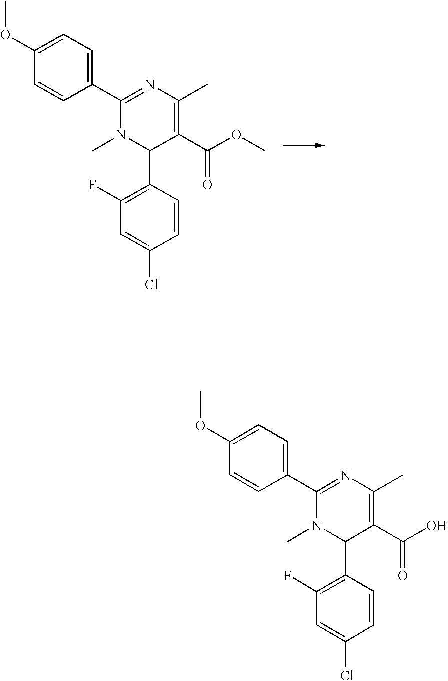

To a solution of the product from above reaction (150 mg, 0.37 mmol) in methanol (5 mL) was added aqueous NaOH (1N, 5 mL). The reaction mixture was heated at 50° C. for two days. The reaction mixture was diluted with water and extracted with ethyl acetate. The organic phase was washed again with 1N NaOH. The aqueous phases were combined and acidified to pH˜1 with 5N HCl. The aqueous solution was extracted twice with ethyl acetate. The combined organic extracts were washed with saturated NaCl, dried over Na₂SO₄, and filtered. The filtrate was concentrated in vacuo and azeotroped several times with hexane. The resulting solid was triturated with 50% CH₂Cl₂/hexanes to afford 100 mg of the acid (69%) as an off-white solid. MS (ES+) m/z 389 [M+H]⁺.

### Step 4. 6-(4-Chloro-2-fluorophenyl)-N-1H-indazol-5-yl-1,4-dimethyl-2-[4-(methyloxy)phenyl]-1,6-dihydro-5-pyrimidinecarboxamide

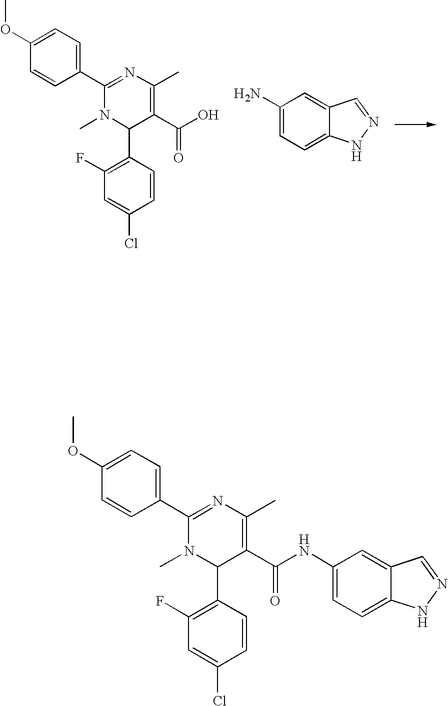

To a solution of the acid from above reaction (100 mg, 0.26 mmol), 5-aminoindazole (40 mg, 0.31 mmol), and EDC (60 mg, 0.31 mmol) in DMF (anhydrous, 2 mL) was added triethylamine (0.02 mL, 0.14 mmol). The reaction mixture was heated at 60° C. for 16 hours. The product was purified by HPLC (RP, 10-90% CH₃CN/H₂O with 0.1% TFA) to afford the title compound (5 mg, 4%). MS (ES+) m/z 504 [M+H]⁺.

Examples 3-122 in the table below were made in a similar manner as in Example 1 and 2.

| # | Structure | Compound Name | m/z |
|---|---|---|---|
| 3 | 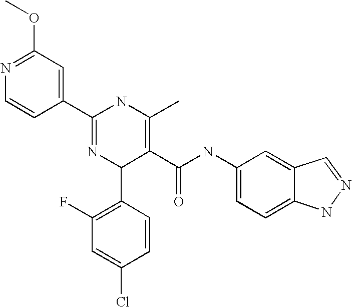 | 4-(4-chloro-2-fluorophenyl)-N-1H-indazol-5-yl-6-methyl-2-[2-(methyloxy)-4-pyridinyl]-1,4-dihydro-5-pyrimidinecarboxamide | 491 |
| 4 | 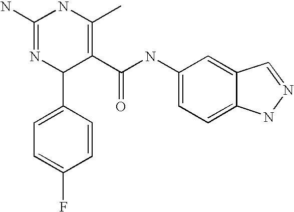 | 2-amino-4-(4-fluorophenyl)-N-1H-indazol-5-yl-6-methyl-1,4-dihydro-5-pyrimidinecarboxamide | 365 |
| 5 | 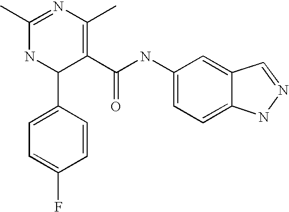 | 4-(4-fluorophenyl)-N-1H-indazol-5-yl-2,6-dimethyl-1,4-dihydro-5-pyrimidinecarboxamide | 364 |
| 6 | 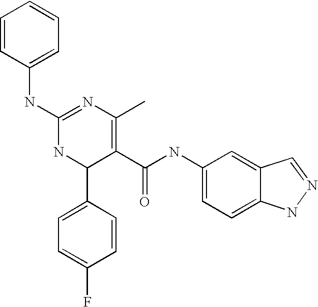 | 4-(4-fluorophenyl)-N-1H-indazol-5-yl-6-methyl-2-(phenylamino)-1,4-dihydro-5-pyrimidinecarboxamide | 441 |
| 7 | 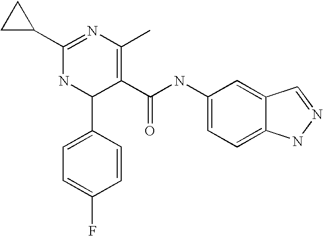 | 2-cyclopropyl-4-(4-fluorophenyl)-N-1H-indazol-5-yl-6-methyl-1,4-dihydro-5-pyrimidinecarboxamide | 390 |
| 8 | 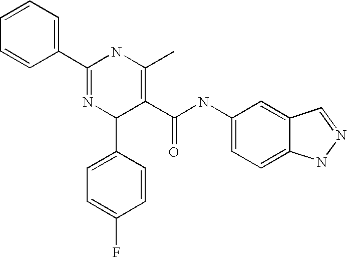 | 4-(4-fluorophenyl)-N-1H-indazol-5-yl-6-methyl-2-phenyl-1,4-dihydro-5-pyrimidinecarboxamide | 426 |
| 9 | 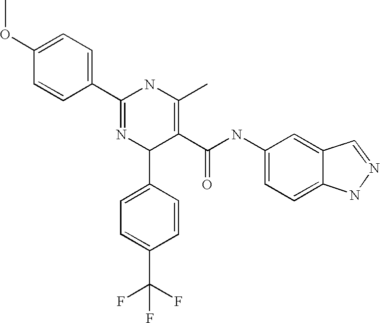 | N-1H-indazol-5-yl-2-(4-methoxyphenyl)-6-methyl-4-[4-(trifluoromethyl)phenyl]-1,4-dihydropyrimidine-5-carboxamide | 506 |
| 10 | 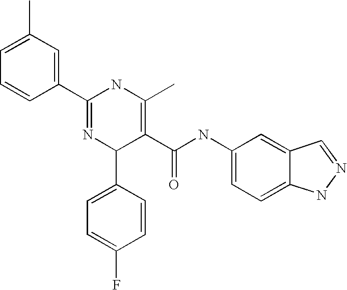 | 4-(4-fluorophenyl)-N-1H-indazol-5-yl-6-methyl-2-(3-methylphenyl)-1,4-dihydro-5-pyrimidinecarboxamide | 440 |
| 11 | 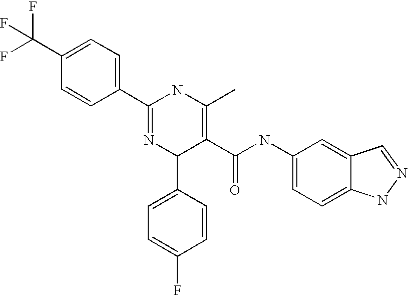 | 4-(4-fluorophenyl)-N-1H-indazol-5-yl-6-methyl-2-[4-(trifluoromethyl)phenyl]-1,4-dihydro-5-pyrimidinecarboxamide | 494 |
| 12 | 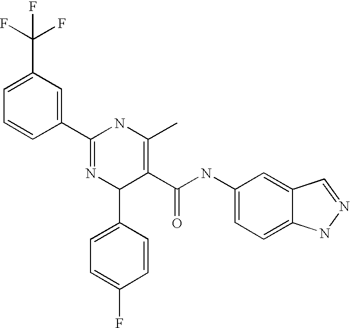 | 4-(4-fluorophenyl)-N-1H-indazol-5-yl-6-methyl-2-[3-(trifluoromethyl)phenyl]-1,4-dihydro-5-pyrimidinecarboxamide | 494 |
| 13 | 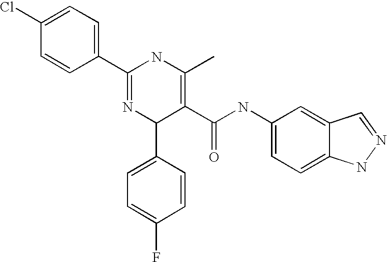 | 2-(4-chlorophenyl)-4-(4-fluorophenyl)-N-1H-indazol-5-yl-6-methyl-1,4-dihydro-5-pyrimidinecarboxamide | 460 |
| 14 | 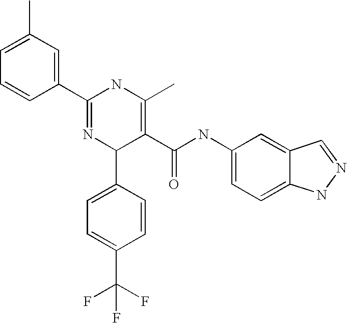 | N-1H-indazol-5-yl-4-methyl-2-(3-methylphenyl)-6-[4-(trifluoromethyl)phenyl]-1,6-dihydro-5-pyrimidinecarboxamide | 490 |
| 15 | 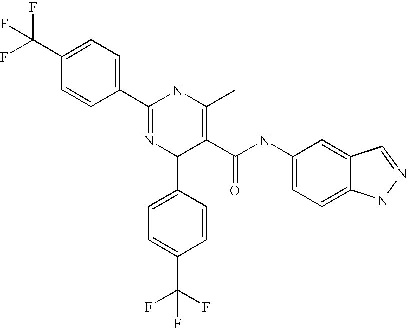 | N-1H-indazol-5-yl-4-methyl-2,6-bis[4-(trifluoromethyl)phenyl]-1,6-dihydro-5-pyrimidinecarboxamide | 544 |
| 16 | 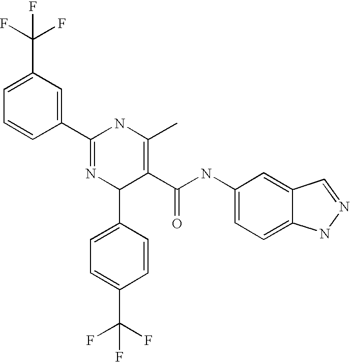 | N-1H-indazol-5-yl-4-methyl-2-[3-(trifluoromethyl)phenyl]-6-[4-(trifluoromethyl)phenyl]-1,6-dihydro-5-pyrimidinecarboxamide | 544 |
| 17 | 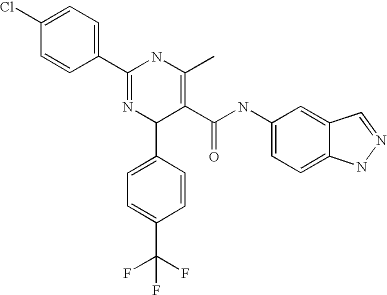 | 2-(4-chlorophenyl)-N-1H-indazol-5-yl-4-methyl-6-[4-(trifluoromethyl)phenyl]-1,6-dihydro-5-pyrimidinecarboxamide | 510 |
| 18 | 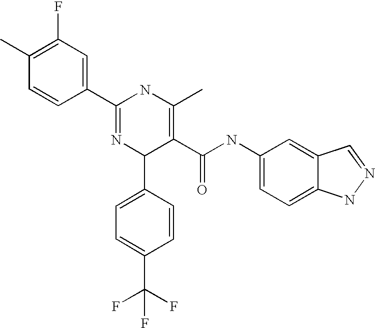 | 2-(3-fluoro-4-methylphenyl)-N-1H-indazol-5-yl-4-methyl-6-[4-(trifluoromethyl)phenyl]-1,6-dihydro-5-pyrimidinecarboxamide | 508 |
| 19 | 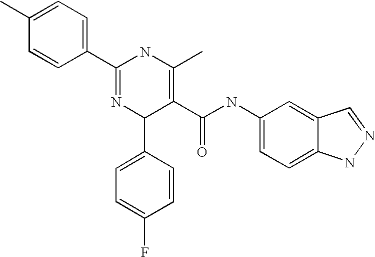 | 4-(4-fluorophenyl)-N-1H-indazol-5-yl-6-methyl-2-(4-methylphenyl)-1,4-dihydro-5-pyrimidinecarboxamide | 440 |
| 20 | 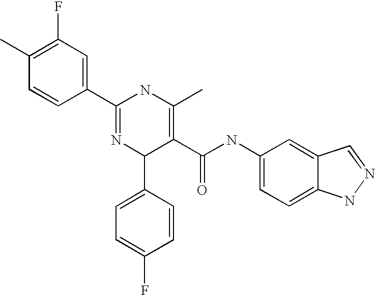 | 2-(3-fluoro-4-methylphenyl)-4-(4-fluorophenyl)-N-1H-indazol-5-yl-6-methyl-1,4-dihydro-5-pyrimidinecarboxamide | 458 |
| 21 | 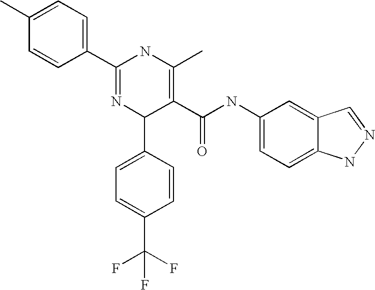 | N-1H-indazol-5-yl-4-methyl-2-(4-methylphenyl)-6-[4-(trifluoromethyl)phenyl]-1,6-dihydro-5-pyrimidinecarboxamide | 490 |
| 22 | 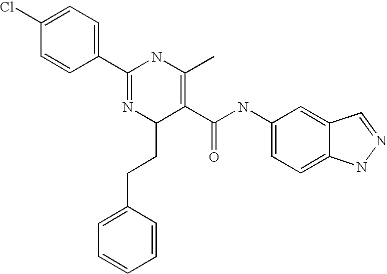 | 2-(4-chlorophenyl)-N-1H-indazol-5-yl-4-methyl-6-(2-phenylethyl)-1,6-dihydro-5-pyrimidinecarboxamide | 470 |
| 23 | 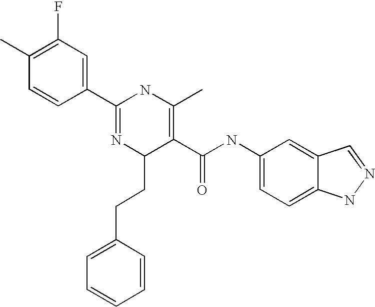 | 2-(3-fluoro-4-methylphenyl)-N-1H-indazol-5-yl-4-methyl-6-(2-phenylethyl)-1,6-dihydro-5-pyrimidinecarboxamide | 468 |
| 24 | 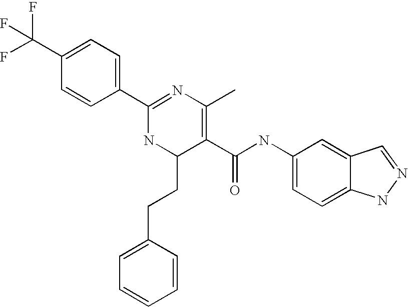 | N-1H-indazol-5-yl-4-methyl-6-(2-phenylethyl)-2-[4-(trifluoromethyl)phenyl]-1,6-dihydro-5-pyrimidinecarboxamide | 504 |
| 25 | 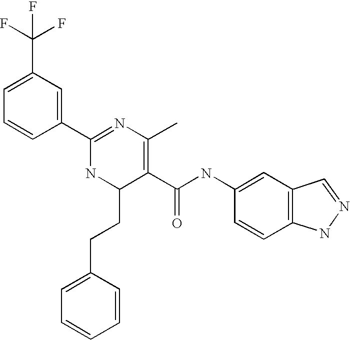 | N-1H-indazol-5-yl-4-methyl-6-(2-phenylethyl)-2-[3-(trifluoromethyl)phenyl]-1,6-dihydro-5-pyrimidinecarboxamide | 504 |
| 26 | 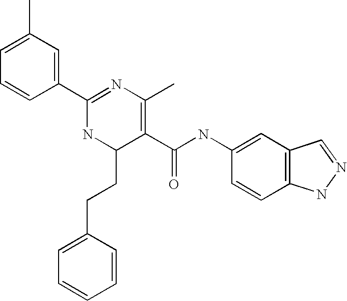 | N-1H-indazol-5-yl-4-methyl-2-(3-methylphenyl)-6-(2-phenylethyl)-1,6-dihydro-5-pyrimidinecarboxamide | 450 |
| 27 | 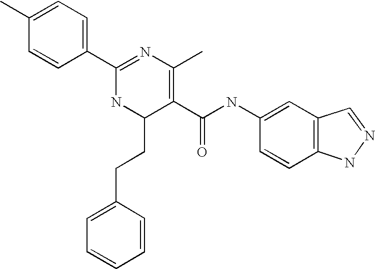 | N-1H-indazol-5-yl-4-methyl-2-(4-methylphenyl)-6-(2-phenylethyl)-1,6-dihydro-5-pyrimidinecarboxamide | 450 |
| 28 | 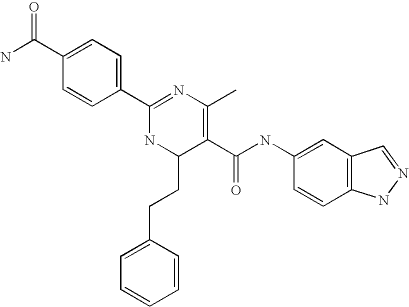 | 2-[4-(aminocarbonyl)phenyl]-N-1H-indazol-5-yl-4-methyl-6-(2-phenylethyl)-1,6-dihydro-5-pyrimidinecarboxamide | 479 |
| 29 | 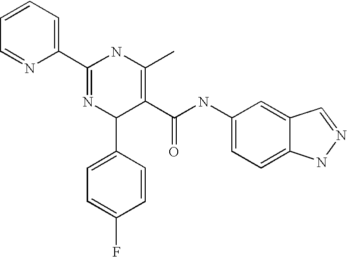 | 4-(4-fluorophenyl)-N-1H-indazol-5-yl-6-methyl-2-(2-pyridinyl)-1,4-dihydro-5-pyrimidinecarboxamide | 427 |
| 30 | 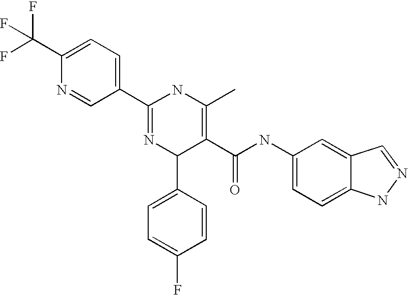 | 4-(4-fluorophenyl)-N-1H-indazol-5-yl-6-methyl-2-[6-(trifluoromethyl)-3-pyridinyl]-1,4-dihydro-5-pyrimidinecarboxamide | 495 |
| 31 | 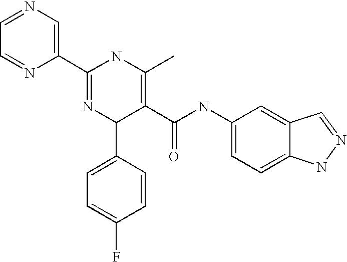 | 4-(4-fluorophenyl)-N-1H-indazol-5-yl-6-methyl-2-(2-pyrazinyl)-1,4-dihydro-5-pyrimidinecarboxamide | 428 |
| 32 | 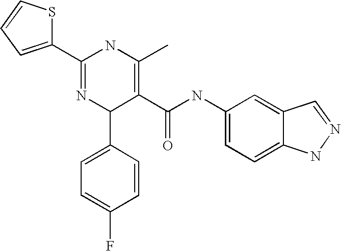 | 4-(4-fluorophenyl)-N-1H-indazol-5-yl-6-methyl-2-(2-thienyl)-1,4-dihydro-5-pyrimidinecarboxamide | 432 |
| 33 | 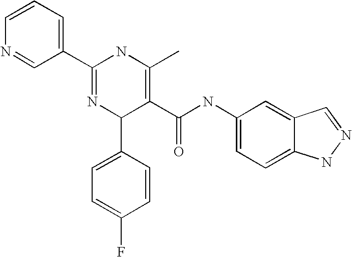 | 4-(4-fluorophenyl)-N-1H-indazol-5-yl-6-methyl-2-(3-pyridinyl)-1,4-dihydro-5-pyrimidinecarboxamide | 427 |
| 34 | 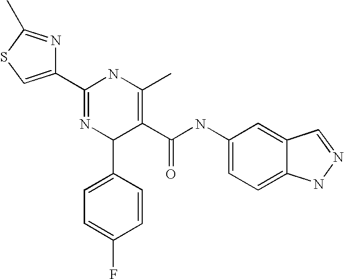 | 4-(4-fluorophenyl)-N-1H-indazol-5-yl-6-methyl-2-(2-methyl-1,3-thiazol-4-yl)-1,4-dihydro-5-pyrimidinecarboxamide | 447 |
| 35 | 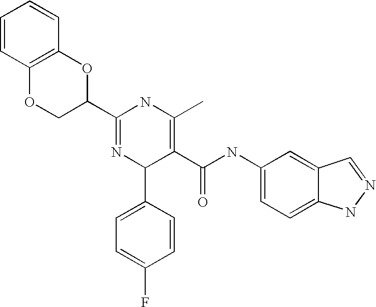 | 2-(2,3-dihydro-1,4-benzodioxiN-2-yl)-4-(4-fluorophenyl)-N-1H-indazol-5-yl-6-methyl-1,4-dihydro-5-pyrimidinecarboxamide | 484 |
| 36 | 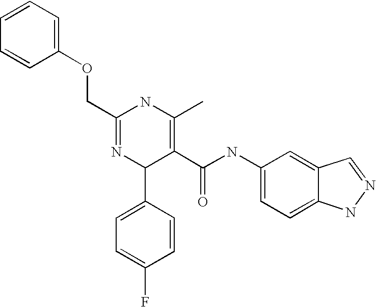 | 4-(4-fluorophenyl)-N-1H-indazol-5-yl-6-methyl-2-[(phenyloxy)methyl]-1,4-dihydro-5-pyrimidinecarboxamide | 456 |
| 37 | 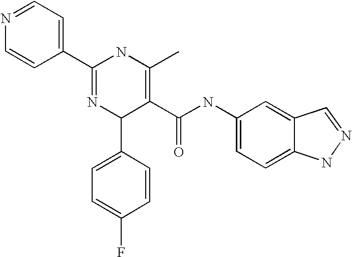 | 4-(4-fluorophenyl)-N-1H-indazol-5-yl-6-methyl-2-(4-pyridinyl)-1,4-dihydro-5-pyrimidinecarboxamide | 427 |
| 38 | 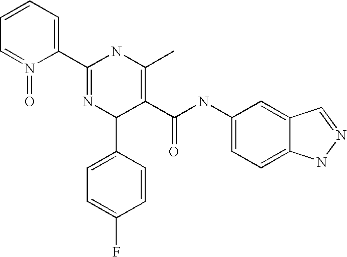 | 4-(4-fluorophenyl)-N-1H-indazol-5-yl-6-methyl-2-(1-oxido-2-pyridinyl)-1,4-dihydro-5-pyrimidinecarboxamide | 443 |
| 39 | 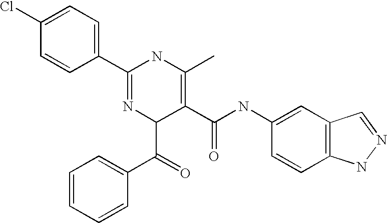 | 2-(4-chlorophenyl)-N-1H-indazol-5-yl-4-methyl-6-(phenylcarbonyl)-1,6-dihydro-5-pyrimidinecarboxamide | 470 |
| 40 | 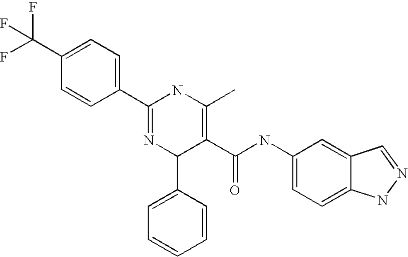 | N-1H-indazol-5-yl-4-methyl-6-phenyl-2-[4-(trifluoromethyl)phenyl]-1,6-dihydro-5-pyrimidinecarboxamide | 476 |
| 41 | 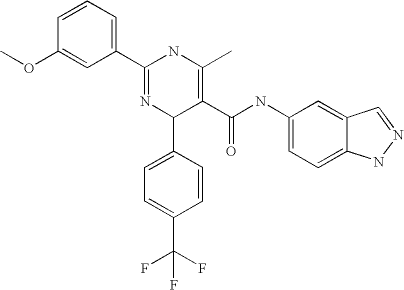 | N-1H-indazol-5-yl-4-methyl-2-[3-(methyloxy)phenyl]-6-[4-(trifluoromethyl)phenyl]-1,6-dihydro-5-pyrimidinecarboxamide | 506 |
| 42 | 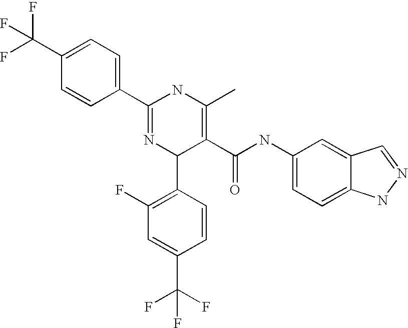 | 4-[2-fluoro-4-(trifluoromethyl)phenyl]-N-1H-indazol-5-yl-6-methyl-2-[4-(trifluoromethyl)phenyl]-1,4-dihydro-5-pyrimidinecarboxamide | 562 |
| 43 | 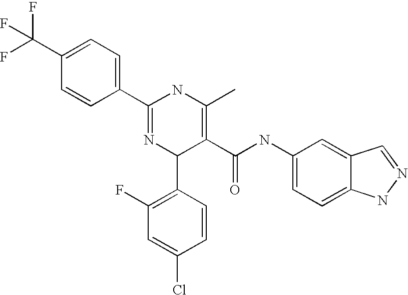 | 4-(4-chloro-2-fluorophenyl)-N-1H-indazol-5-yl-6-methyl-2-[4-(trifluoromethyl)phenyl]-1,4-dihydro-5-pyrimidinecarboxamide | 528 |
| 44 | 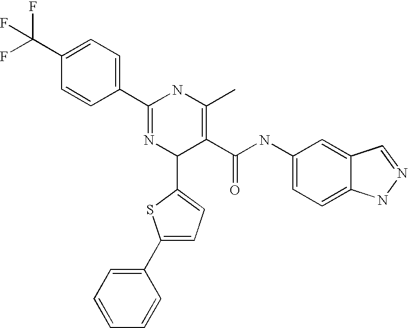 | N-1H-indazol-5-yl-4-methyl-6-(5-phenyl-2-thienyl)-2-[4-(trifluoromethyl)phenyl]-1,6-dihydro-5-pyrimidinecarboxamide | 558 |
| 45 | 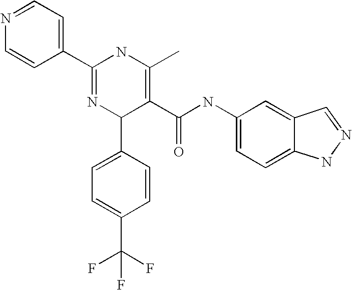 | N-1H-indazol-5-yl-4-methyl-2-(4-pyridinyl)-6-[4-(trifluoromethyl)phenyl]-1,6-dihydro-5-pyrimidinecarboxamide | 477 |
| 46 | 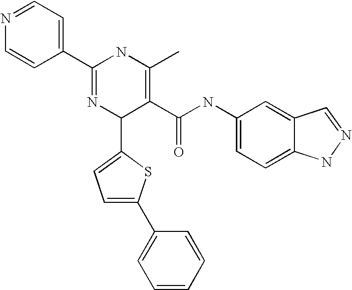 | N-1H-indazol-5-yl-4-methyl-6-(5-phenyl-2-thienyl)-2-(4-pyridinyl)-1,6-dihydro-5-pyrimidinecarboxamide | 491 |
| 47 | 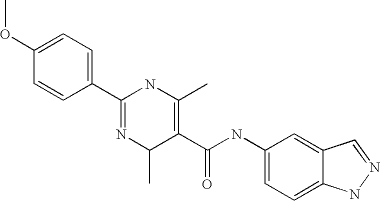 | N-1H-indazol-5-yl-4,6-dimethyl-2-[4-(methyloxy)phenyl]-1,4-dihydro-5-pyrimidinecarboxamide | 376 |
| 48 | 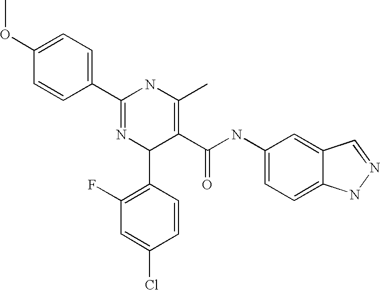 | 4-(4-chloro-2-fluorophenyl)-N-1H-indazol-5-yl-6-methyl-2-[4-(methyloxy)phenyl]-1,4-dihydro-5-pyrimidinecarboxamide | 490 |
| 49 | 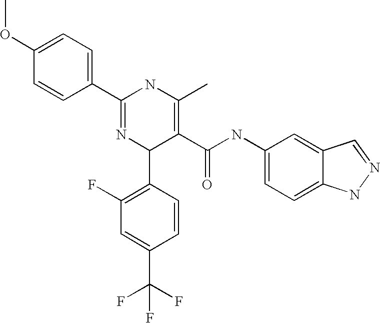 | 4-[2-fluoro-4-(trifluoromethyl)phenyl]-N-1H-indazol-5-yl-6-methyl-2-[4-(methyloxy)phenyl]-1,4-dihydro-5-pyrimidinecarboxamide | 524 |
| 50 | 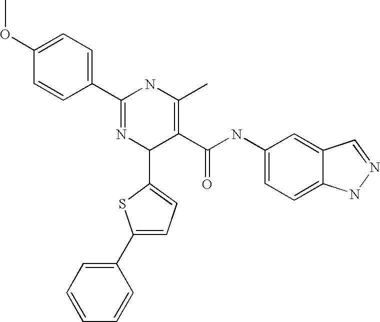 | N-1H-indazol-5-yl-4-methyl-2-[4-(methyloxy)phenyl]-6-(5-phenyl-2-thienyl)-1,6-dihydro-5-pyrimidinecarboxamide | 520 |
| 51 | 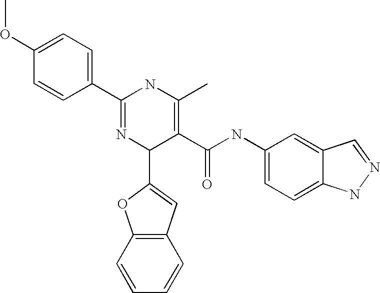 | 4-(1-benzofuraN-2-yl)-N-1H-indazol-5-yl-6-methyl-2-[4-(methyloxy)phenyl]-1,4-dihydro-5-pyrimidinecarboxamide | 478 |
| 52 | 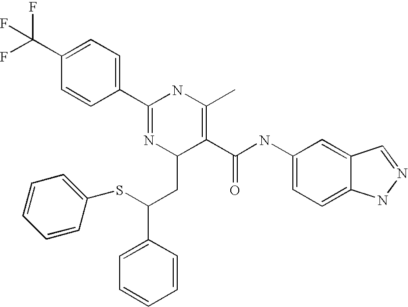 | N-1H-indazol-5-yl-4-methyl-6-[2-phenyl-2-(phenylthio)ethyl]-2-[4-(trifluoromethyl)phenyl]-1,6-dihydro-5-pyrimidinecarboxamide | 612 |
| 53 | 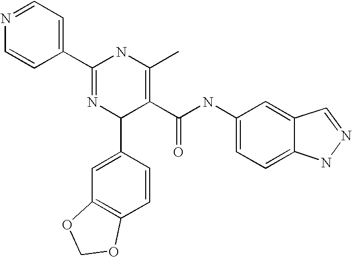 | 4-(1,3-benzodioxol-5-yl)-N-1H-indazol-5-yl-6-methyl-2-(4-pyridinyl)-1,4-dihydro-5-pyrimidinecarboxamide | 453 |
| 54 | 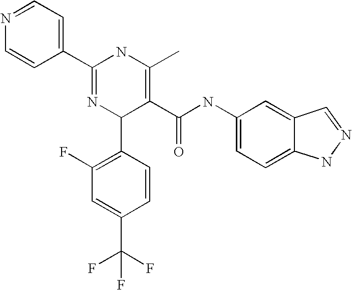 | 4-[2-fluoro-4-(trifluoromethyl)phenyl]-N-1H-indazol-5-yl-6-methyl-2-(4-pyridinyl)-1,4-dihydro-5-pyrimidinecarboxamide | 495 |
| 55 | 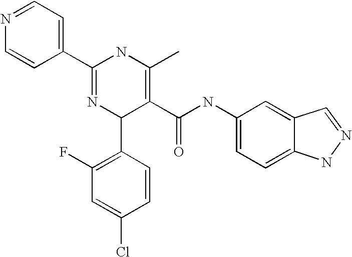 | 4-(4-chloro-2-fluorophenyl)-N-1H-indazol-5-yl-6-methyl-2-(4-pyridinyl)-1,4-dihydro-5-pyrimidinecarboxamide | 461 |
| 56 | 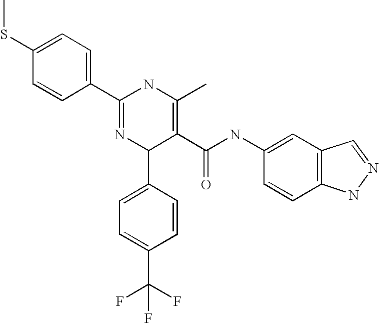 | N-1H-indazol-5-yl-4-methyl-2-[4-(methylthio)phenyl]-6-[4-(trifluoromethyl)phenyl]-1,6-dihydro-5-pyrimidinecarboxamide | 522 |
| 57 | 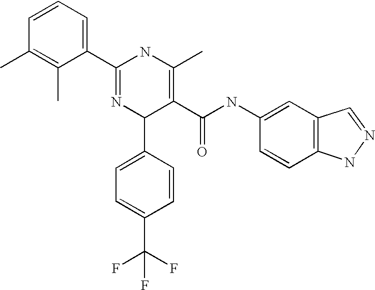 | 2-(2,3-dimethylphenyl)-N-1H-indazol-5-yl-4-methyl-6-[4-(trifluoromethyl)phenyl]-1,6-dihydro-5-pyrimidinecarboxamide | 504 |
| 58 | 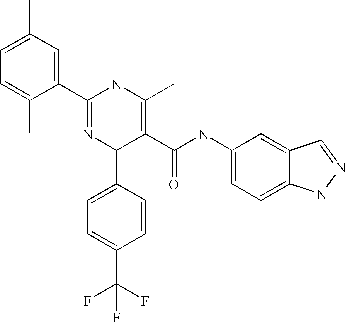 | 2-(2, 5-dimethylphenyl)-N-1H-indazol-5-yl-4-methyl-6-[4-(trifluoromethyl)phenyl]-1,6-dihydro-5-pyrimidinecarboxamide | 504 |
| 59 | 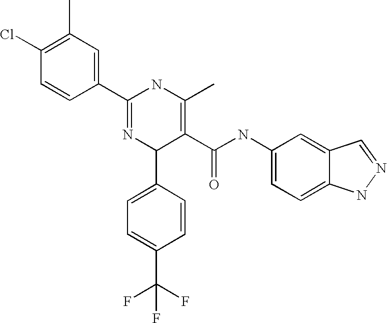 | 2-(4-chloro-3-methylphenyl)-N-1H-indazol-5-yl-4-methyl-6-[4-(trifluoromethyl)phenyl]-1,6-dihydro-5-pyrimidinecarboxamide | 524 |
| 60 | 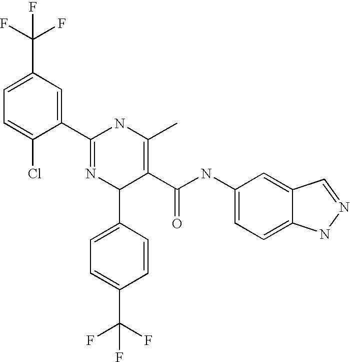 | 2-[2-chloro-5-(trifluoromethyl)phenyl]-N-1H-indazol-5-yl-4-methyl-6-[4-(trifluoromethyl)phenyl]-1,6-dihydro-5-pyrimidinecarboxamide | 578 |
| 61 | 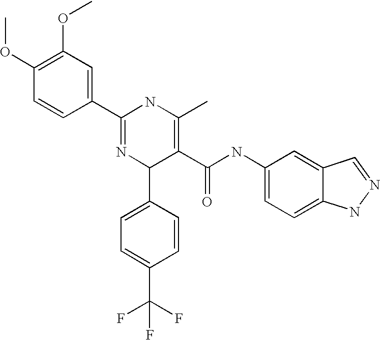 | 2-[3,4-bis(methyloxy)phenyl]-N-1H-indazol-5-yl-4-methyl-6-[4-(trifluoromethyl)phenyl]-1,6-dihydro-5-pyrimidinecarboxamide | 536 |
| 62 | 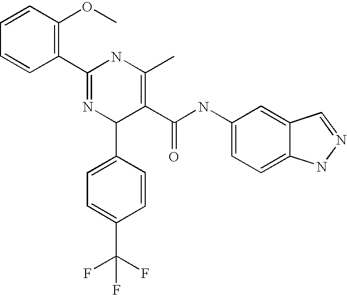 | N-1H-indazol-5-yl-4-methyl-2-[2-(methyloxy)phenyl]-6-[4-(trifluoromethyl)phenyl]-1,6-dihydro-5-pyrimidinecarboxamide | 506 |
| 63 | 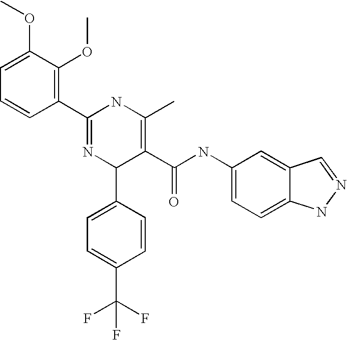 | 2-[2,3-bis(methyloxy)phenyl]-N-1H-indazol-5-yl-4-methyl-6-[4-(trifluoromethyl)phenyl]-1,6-dihydro-5-pyrimidinecarboxamide | 536 |
| 64 | 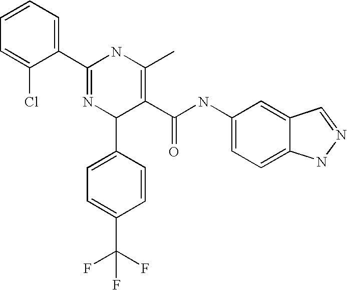 | 2-(2-chlorophenyl)-N-1H-indazol-5-yl-4-methyl-6-[4-(trifluoromethyl)phenyl]-1,6-dihydro-5-pyrimidinecarboxamide | 510 |
| 65 | 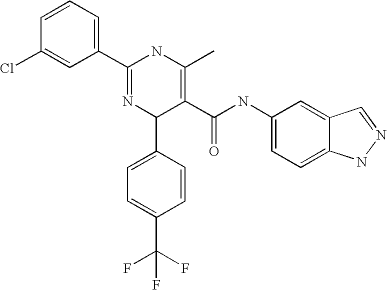 | 2-(3-chlorophenyl)-N-1H-indazol-5-yl-4-methyl-6-[4-(trifluoromethyl)phenyl]-1,6-dihydro-5-pyrimidinecarboxamide | 510 |
| 66 | 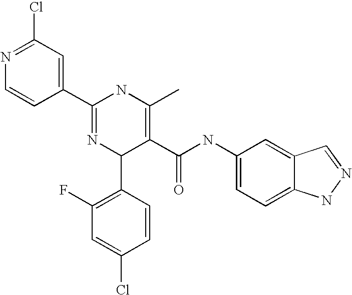 | 4-(4-chloro-2-fluorophenyl)-2-(2-chloro-4-pyridinyl)-N-1H-indazol-5-yl-6-methyl-1,4-dihydro-5-pyrimidinecarboxamide | 496 |
| 67 | 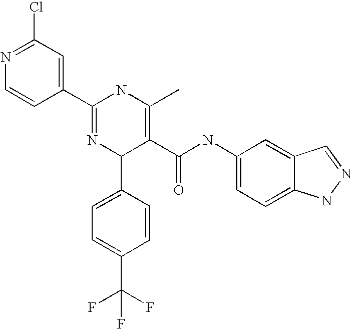 | 2-(2-chloro-4-pyridinyl)-N-1H-indazol-5-yl-4-methyl-6-[4-(trifluoromethyl)phenyl]-1,6-dihydro-5-pyrimidinecarboxamide | 511 |
| 68 | 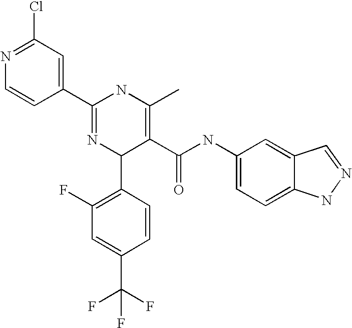 | 2-(2-chloro-4-pyridinyl)-4-[2-fluoro-4-(trifluoromethyl)phenyl]-N-1H-indazol-5-yl-6-methyl-1,4-dihydro-5-pyrimidinecarboxamide | 529 |
| 69 | 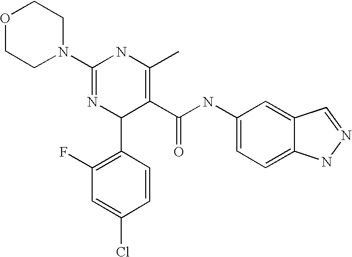 | 4-(4-chloro-2-fluorophenyl)-N-1H-indazol-5-yl-6-methyl-2-(4-morpholinyl)-1,4-dihydro-5-pyrimidinecarboxamide | 469 |
| 70 | 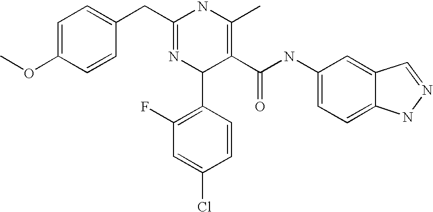 | 4-(4-chloro-2-fluorophenyl)-N-1H-indazol-5-yl-6-methyl-2-{[4-(methyloxy)phenyl]methyl}-1,4-dihydro-5-pyrimidinecarboxamide | 504 |
| 71 | 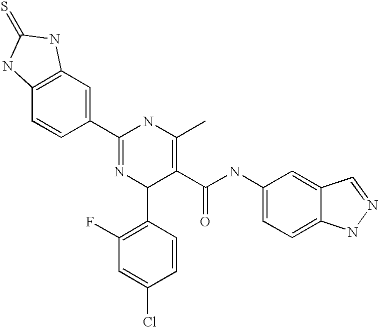 | 4-(4-chloro-2-fluorophenyl)-N-1H-indazol-5-yl-6-methyl-2-(2-thioxo-2,3-dihydro-1H-benzimidazol-5-yl)-1,4-dihydro-5-pyrimidinecarboxamide | 532 |
| 72 | 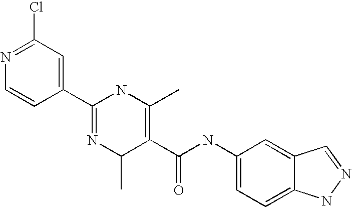 | 2-(2-chloro-4-pyridinyl)-N-1H-indazol-5-yl-4,6-dimethyl-1,4-dihydro-5-pyrimidinecarboxamide | 381 |
| 73 | 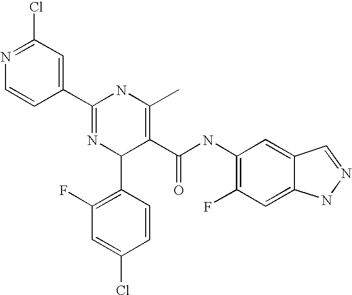 | 4-(4-chloro-2-fluorophenyl)-2-(2-chloro-4-pyridinyl)-N-(6-fluoro-1H-indazol-5-yl)-6-methyl-1,4-dihydro-5-pyrimidinecarboxamide | 514 |
| 74 | 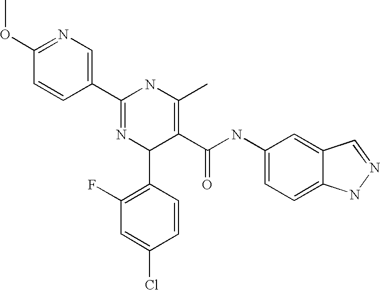 | 4-(4-chloro-2-fluorophenyl)-N-1H-indazol-5-yl-6-methyl-2-[6-(methyloxy)-3-pyridinyl]-1,4-dihydro-5-pyrimidinecarboxamide | 491 |
| 75 | 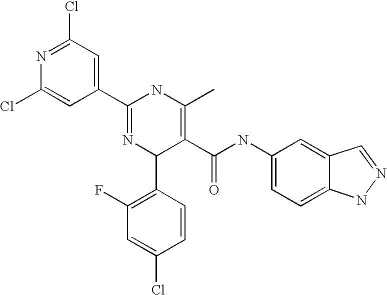 | 4-(4-chloro-2-fluorophenyl)-2-(2,6-dichloro-4-pyridinyl)-N-1H-indazol-5-yl-6-methyl-1,4-dihydro-5-pyrimidinecarboxamide | 530 |
| 76 | 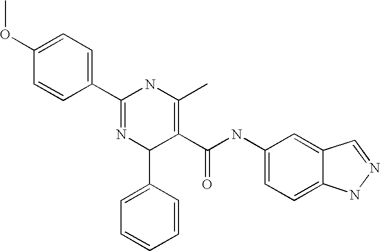 | N-1H-indazol-5-yl-4-methyl-2-[4-(methyloxy)phenyl]-6-phenyl-1,6-dihydro-5-pyrimidinecarboxamide | 438 |
| 77 | 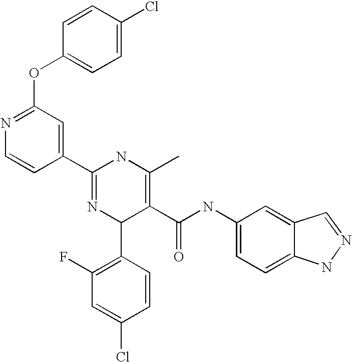 | 4-(4-chloro-2-fluorophenyl)-2-{2-[(4-chlorophenyl)oxy]-4-pyridinyl}-N-1H-indazol-5-yl-6-methyl-1,4-dihydro-5-pyrimidinecarboxamide | 588 |
| 78 | 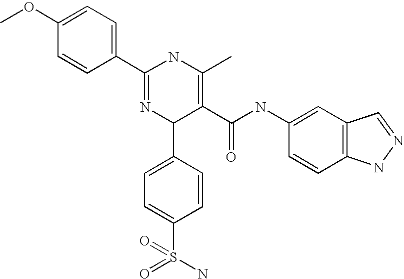 | 4-[4-(aminosulfony)phenyl]-N-1H-indazol-5-yl-6-methyl-2-[4-(methyloxy)phenyl]-1,4-dihydro-5-pyrimidinecarboxamide | 517 |
| 79 | 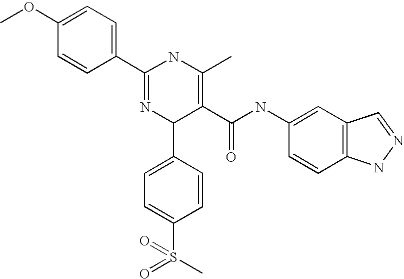 | N-1H-indazol-5-yl-4-methyl-2-[4-(methyloxy)phenyl]-6-[4-(methylsulfonyl)phenyl]-1,6-dihydro-5-pyrimidinecarboxamide | 516 |
| 80 | 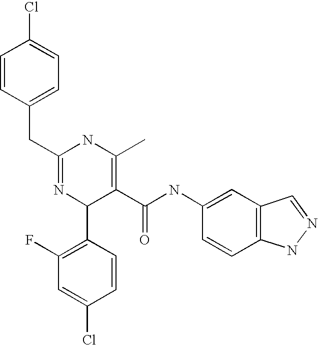 | 4-(4-chloro-2-fluorophenyl)-2-[(4-chlorophenyl)methyl]-N-1H-indazol-5-yl-6-methyl-1,4-dihydro-5-pyrimidinecarboxamide | 509 |
| 81 | 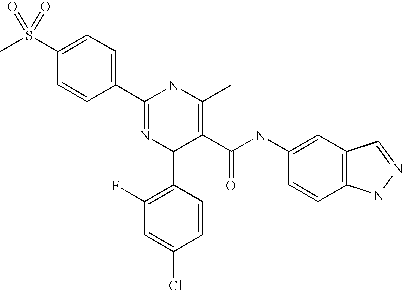 | 4-(4-chloro-2-fluorophenyl)-N-1H-indazol-5-yl-6-methyl-2-(methylsulfonyl)-1,4-dihydro-5-pyrimidinecarboxamide | 538 |
| 82 | 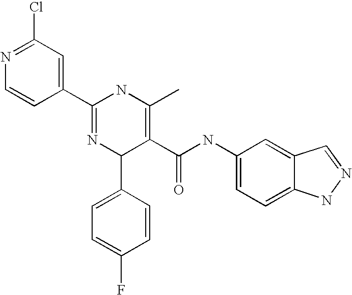 | 2-(2-chloro-4-pyridinyl)-4-(4-fluorophenyl)-N-1H-indazol-5-yl-methyl-1,4-dihydro-5-pyrimidinecarboxamide | 461 |
| 83 | 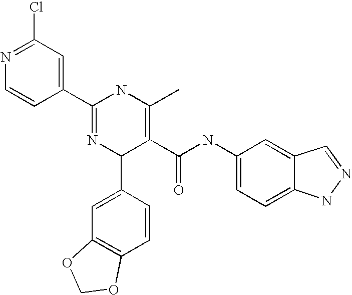 | 4-(1,3-benzodioxol-5-yl)-2-(2-chloro-4-pyridinyl)-N-1H-indazol-5-yl-6-methyl-1,4-dihydro-5-pyrimidinecarboxamide | 487 |
| 84 | 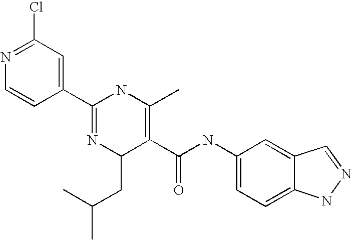 | 2-(2-chloro-4-pyridinyl)-N-1H-indazol-5-yl-4-methyl-6-(2-methylpropyl)-1,6-dihydro-5-pyrimidinecarboxamide | 423 |
| 85 | 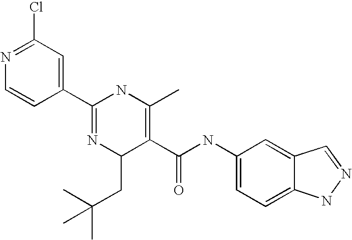 | 2-(2-chloro-4-pyridinyl)-4-(2,2-dimethylpropyl)-N-1H-indazol-5-yl-6-methyl-1,4-dihydro-5-pyrimidinecarboxamide | 437 |
| 86 | 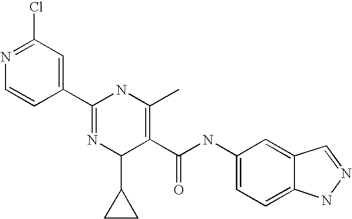 | 2-(2-chloro-4-pyridinyl)-4-cyclopropyl-N-1H-indazol-5-yl-6-methyl-1,4-dihydro-5-pyrimidinecarboxamide | 407 |
| 87 | 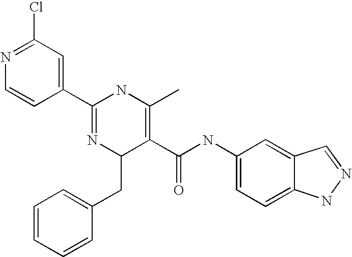 | 2-(2-chloro-4-pyridinyl)-N-1H-indazol-5-yl-4-methyl-6-(phenylmethyl)-1,6-dihydro-5-pyrimidinecarboxamide | 457 |
| 88 | 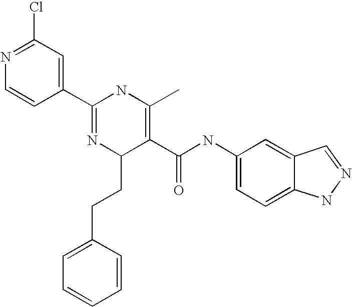 | 2-(2-chloro-4-pyridinyl)-N-1H-indazol-5-yl-4-methyl-6-(2-phenylethyl)-1,6-dihydro-5-pyrimidinecarboxamide | 471 |
| 89 | 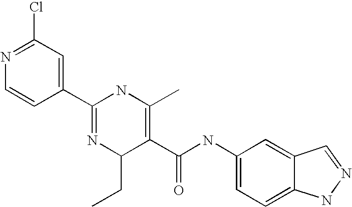 | 2-(2-chloro-4-pyridinyl)-4-ethyl-N-1H-indazol-5-yl-6-methyl-1,4-dihydro-5-pyrimidinecarboxamide | 395 |
| 90 | 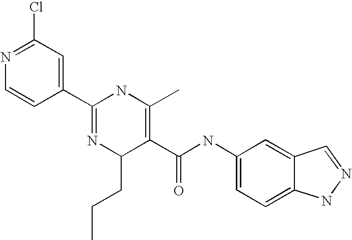 | 2-(2-chloro-4-pyridinyl)-N-1H-indazol-5-yl-4-methyl-6-propyl-1,6-dihydro-5-pyrimidinecarboxamide | 409 |
| 91 | 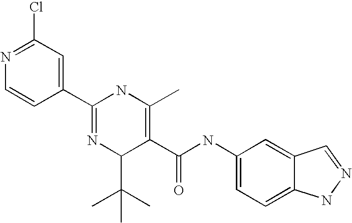 | 2-(2-chloro-4-pyridinyl)-4-(1,1-dimethylethyl)-N-1H-indazol-5-yl-6-methyl-1,4-dihydro-5-pyrimidinecarboxamide | 423 |
| 92 | 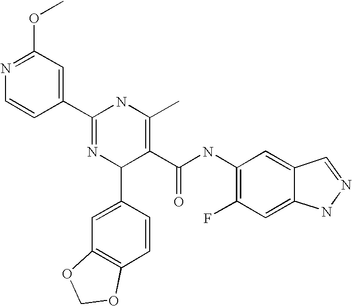 | 4-(1,3-benzodioxol-5-yl)-N-(6-fluoro-1H-indazol-5-yl)-6-methyl-2-[2-(methyloxy)-4-pyridinyl]-1,4-dihydro-5-pyrimidinecarboxamide | 501 |
| 93 | 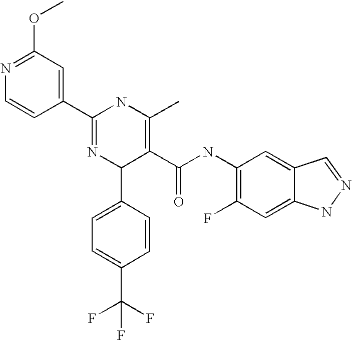 | N-(6-fluoro-1H-indazol-5-yl)-4-methyl-2-[2-(methyloxy)-4-pyridinyl]-6-[4-(trifluoromethyl)phenyl]-1,6-dihydro-5-pyrimidinecarboxamide | 525 |
| 94 | 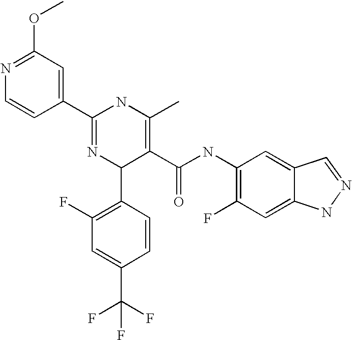 | N-(6-fluoro-1H-indazol-5-yl)-4-[2-fluoro-4-(trifluoromethyl)phenyl]-6-methyl-2-[2-(methyloxy)-4-pyridinyl]-1,4-dihydro-5-pyrimidinecarboxamide | 543 |
| 95 | 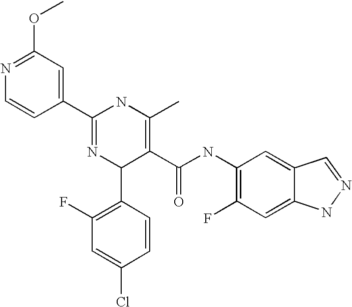 | 4-(4-chloro-2-fluorophenyl)-N-(6-fluoro-1H-indazol-5-yl)-6-methyl-2-[2-(methyloxy)-4-pyridinyl]-1,4-dihydro-5-pyrimidinecarboxamide | 509 |
| 96 | 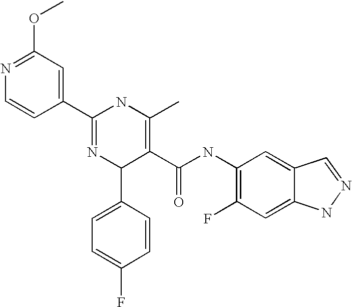 | N-(6-fluoro-1H-indazol-5-yl)-4-(4-fluorophenyl)-6-methyl-2-[2-(methyloxy)-4-pyridinyl]-1,4-dihydro-5-pyrimidinecarboxamide | 475 |
| 97 | 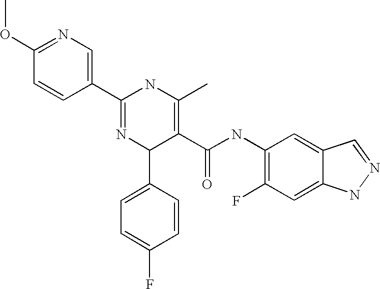 | N-(6-fluoro-1H-indazol-5-yl)-4-(4-fluorophenyl)-6-methyl-2-[6-(methyloxy)-3-pyridinyl]-1,4-dihydro-5-pyrimidinecarboxamide | 475 |
| 98 | 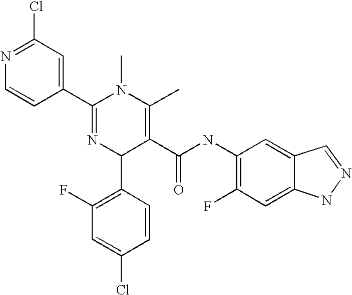 | 4-(4-chloro-2-fluorophenyl)-2-(2-chloro-4-pyridinyl)-N-(6-fluoro-1H-indazol-5-yl)-1,6-dimethyl-1,4-dihydro-5-pyrimidinecarboxamide | 528 |
| 99 | 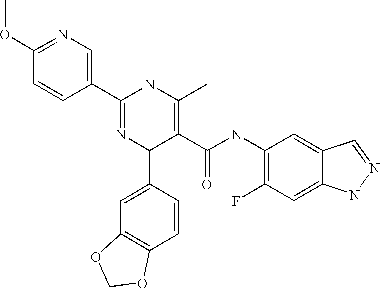 | 4-(1,3-benzodioxol-5-yl)-N-(6-fluoro-1H-indazol-5-yl)-6-methyl-2-[6-(methyloxy)-3-pyridinyl]-1,4-dihydro-5-pyrimidinecarboxamide | 501 |
| 100 | 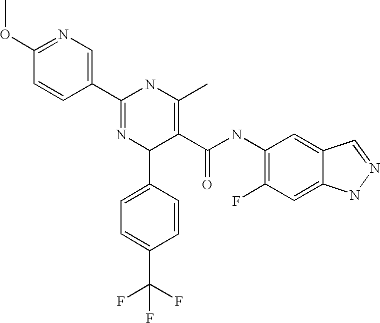 | N-(6-fluoro-1H-indazol-5-yl)-4-methyl-2-[6-(methyloxy)-3-pyridinyl]-6-[4-(trifluoromethyl)phenyl]-1,6-dihydro-5-pyrimidinecarboxamide | 525 |
| 101 | 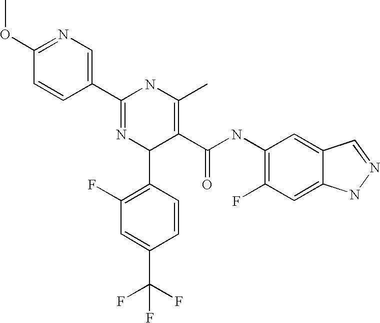 | N-(6-fluoro-1H-indazol-5-yl)-4-[2-fluoro-4-(trifluoromethyl)phenyl]-6-methyl-2-[6-(methyloxy)-3-pyridinyl]-1,4-dihydro-5-pyrimidinecarboxamide | 543 |
| 102 | 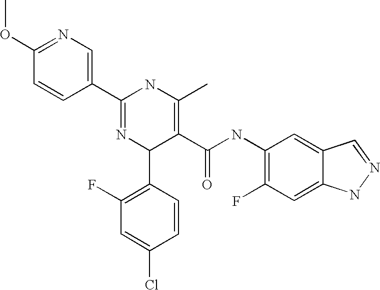 | 4-(4-chloro-2-fluorophenyl)-N-(6-fluoro-1H-indazol-5-yl)-6-methyl-2-[6-(methyloxy)-3-pyridinyl]-1,4-dihydro-5-pyrimidinecarboxamide | 509 |
| 103 | 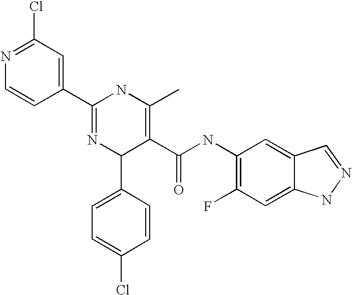 | 4-(4-chlorophenyl)-2-(2-chloro-4-pyridinyl)-N-(6-fluoro-1H-indazol-5-yl)-6-methyl-1,4-dihydro-5-pyrimidinecarboxamide | 496 |
| 104 | 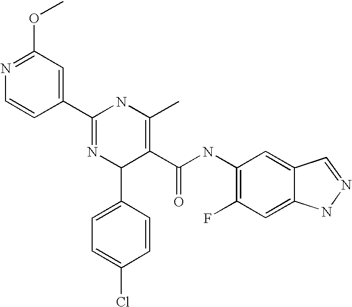 | 4-(4-chlorophenyl)-N-(6-fluoro-1H-indazol-5-yl)-6-methyl-2-[2-(methyloxy)-4-pyridinyl]-1,4-dihydro-5-pyrimidinecarboxamide | 491 |
| 105 | 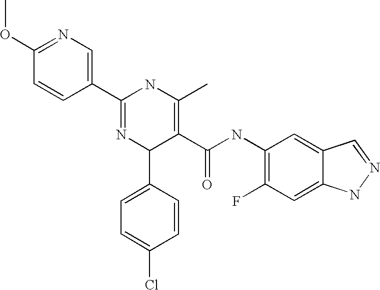 | 4-(4-chlorophenyl)-N-(6-fluoro-1H-indazol-5-yl)-6-methyl-2-[6-(methyloxy)-3-pyridinyl]-1,4-dihydro-5-pyrimidinecarboxamide | 491 |
| 106 | 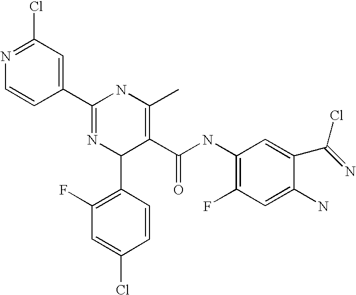 | N-(3-chloro-6-fluoro-1H-indazol-5-yl)-4-(4-chloro-2-fluorophenyl)-2-(2-chloro-4-pyridinyl)-6-methyl-1,4-dihydro-5-pyrimidinecarboxamide | 548 |
| 107 | 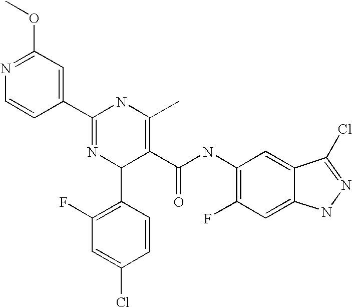 | N-(3-chloro-6-fluoro-1H-indazol-5-yl)-4-(4-chloro-2-fluorophenyl)-6-methyl-2-[2-(methyloxy)-4-pyridinyl]-1,4-dihydro-5-pyrimidinecarboxamide | 544 |
| 108 | 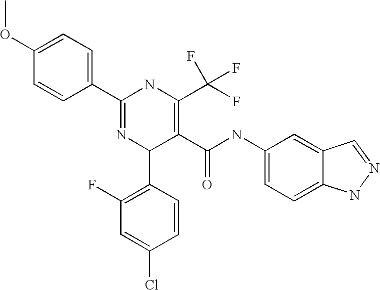 | 4-(4-chloro-2-fluorophenyl)-N-1H-indazol-5-yl-2-[4-(methyloxy)phenyl]-6-(trifluoromethyl)-1,4-dihydro-5-pyrimidinecarboxamide | 544 |
| 109 | 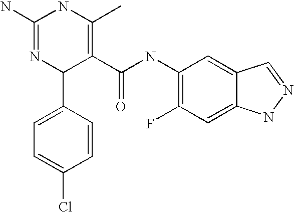 | 2-amino-4-(4-chlorophenyl)-N-(6-fluoro-1H-indazol-5-yl)-6-methyl-1,4-dihydro-5-pyrimidinecarboxamide | 399 |
| 110 | 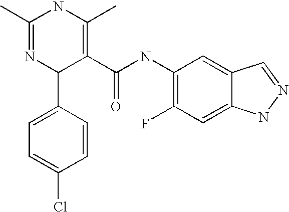 | 4-(4-chlorophenyl)-N-(6-fluoro-1H-indazol-5-yl)-2,6-dimethyl-1,4-dihydro-5-pyrimidinecarboxamide | 398 |
| 111 | 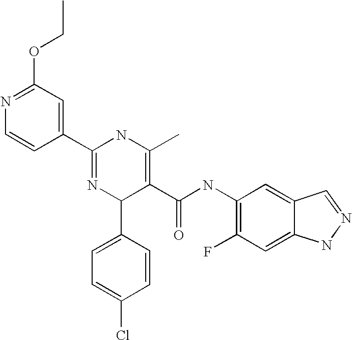 | 4-(4-chlorophenyl)-2-[2-(ethyloxy)-4-pyridinyl]-N-(6-fluoro-1H-indazol-5-yl)-6-methyl-1,4-dihydro-5-pyrimidinecarboxamide | 505 |
| 112 | 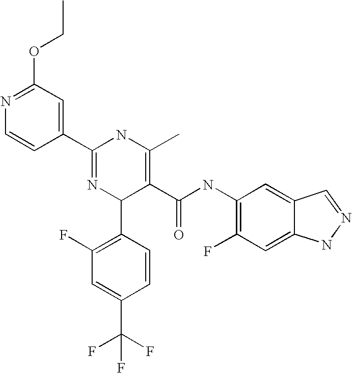 | 2-[2-(ethyloxy)-4-pyridinyl]-N-(6-fluoro-1H-indazol-5-yl)-4-[2-fluoro-4-(trifluoromethyl)phenyl]-6-methyl-1,4-dihydro-5-pyrimidinecarboxamide | 557 |
| 113 | 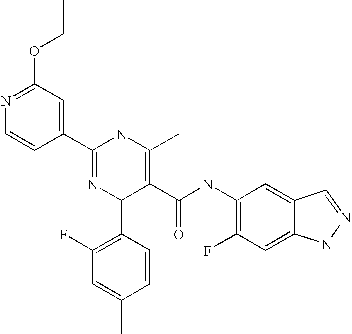 | 4-(4-chloro-2-fluorophenyl)-2-[2-(ethyloxy)-4-pyridinyl]-N-(6-fluoro-1H-indazol-5-yl)-6-methyl-1,4-dihydro-5-pyrimidinecarboxamide | 523 |
| 114 | 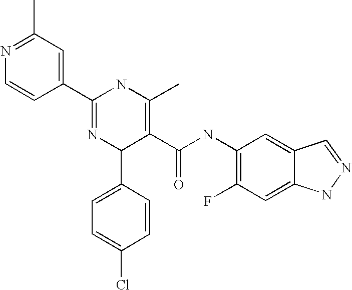 | 4-(4-chlorophenyl)-N-(6-fluoro-1H-indazol-5-yl)-6-methyl-2-(2-methyl-4-pyridinyl)-1,4-dihydro-5-pyrimidinecarboxamide | 475 |
| 115 | 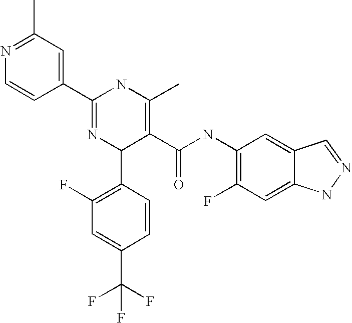 | N-(6-fluoro-1H-indazol-5-yl)-4-[2-fluoro-4-(trifluoromethyl)phenyl]-6-methyl-2-(2-methyl-4-pyridinyl)-1,4-dihydro-5-pyrimidinecarboxamide | 527 |
| 116 | 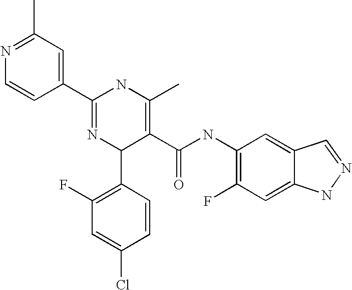 | 4-(4-chloro-2-fluorophenyl)-N-(6-fluoro-1H-indazol-5-yl)-6-methyl-2-(2-methyl-4-pyridinyl)-1,4-dihydro-5-pyrimidinecarboxamide | 493 |
| 117 | 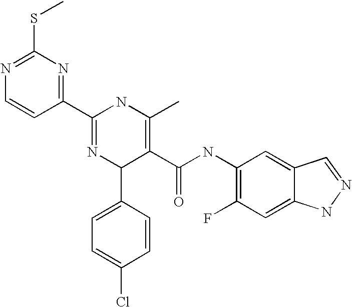 | 4-(4-chlorophenyl)-N-(6-fluoro-1H-indazol-5-yl)-6-methyl-2′-(methylthio)-1,4-dihydro-2,4′-bipyrimidine-5-carboxamide | 508 |
| 118 | 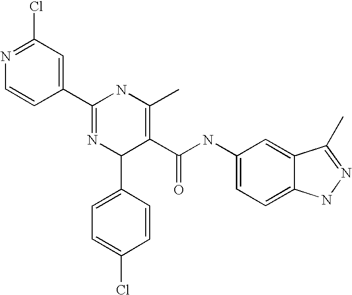 | 4-(4-chlorophenyl)-2-(2-chloro-4-pyridinyl)-6-methyl-N-(3-methyl-1H-indazol-5-yl)-1,4-dihydro-5-pyrimidinecarboxamide | 492 |
| 119 | 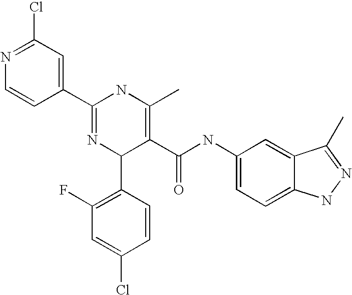 | 4-(4-chloro-2-fluorophenyl)-2-(2-chloro-4-pyridinyl)-6-methyl-N-(3-methyl-1H-indazol-5-yl)-1,4-dihydro-5-pyrimidinecarboxamide | 510 |
| 120 | 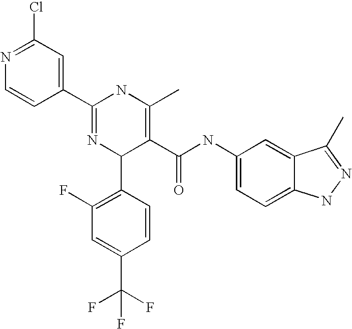 | 2-(2-chloro-4-pyridinyl)-4-[2-fluoro-4-(trifluoromethyl)phenyl]-6-methyl-N-(3-methyl-1H-indazol-5-yl)-1,4-dihydro-5-pyrimidinecarboxamide | 543 |
| 121 | 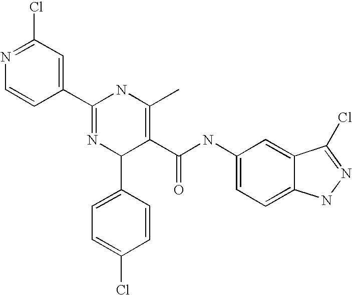 | N-(3-chloro-1H-indazol-5-yl)-4-(4-chlorophenyl)-2-(2-chloro-4-pyridinyl)-6-methyl-1,4-dihydro-5-pyrimidinecarboxamide | 512 |
| 122 | 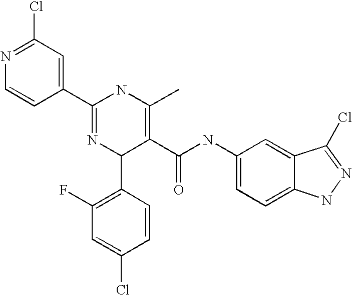 | 4-(4-chloro-2-fluorophenyl)-N-(3-chloro-1H-indazol-5-yl)-2-(2-chloro-4-pyridinyl)-6-methyl-1,4-dihydro-5-pyrimidinecarboxamide | 530 |

### Preparation of Selected Intermediates

### 2-(Methyloxy)-4-pyridinecarbonitrile

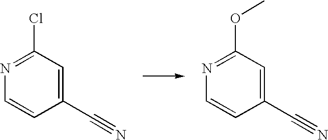

To a solution of 2-chloro-4-cyanopyridine (1.38 g, 10 mmol) in dioxane (10 mL) was added a solution of sodium methoxide in methanol (25% w, 2.27 g, 10.5 mmol). The reaction mixture was heated at reflux for 5 hours, then cooled to room temperature and left over night in refrigerator. The precipitate was filtered and washed with methanol. The filtrate was concentrated down to about 10 mL, and water (100 mL) was added. The precipitates was collected by filtration and washed with water to afford the title compound (1.93 g, 72%) as a white solid. MS (ES+) m/z 135 [M+H]⁺.

### 2-Methyl-4-pyridinecarbonitrile

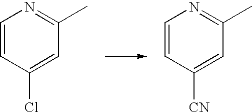

A mixture of 4-chloro-2-methylpyridine (2.55 g, 20 mmol), Pd(OAc)₂ (90 mg, 0.4 mmol), 1,4-bis(diphenylphosphino)butane (341 mg, 0.8 mmol), tetramethylethylenediamine (0.6 mL, 4 mmol), and potassium cyanide (1.3 g, 20 mmol) in toluene (20 mL) was heated at 160° C. for 16 hours, cooled to room temperature, diluted with ethyl acetate (50 mL), washed with water, brine, dried over Na₂SO₄, filtered and concentrated. The residue was purified on silica gel using 10-20% ethyl acetate-hexane to afford the title compound (1.11 g, 47%) as a white solid. MS (ES+) m/z 119 [M+H]⁺.

### 2-(Ethyloxy)-4-pyridinecarbonitrile

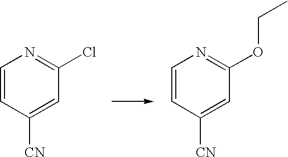

2-Chloro-4-pyridinecarbonitrile (2.76 g, 20 mmol) was added to a suspension of potassium ethoxide (1.77 g, 21 mmol) in dioxane (30 mL) slowly. The resulting mixture was stirred for 2 hours at room temperature, concentrated, and triturated with 20% ethyl acetate-hexane. The solid was filtered, and the filtrate was concentrated, purified on silica gel using 10% ethyl acetate-hexane to afford the title compound (1.16 g, 39%) as a white solid. MS (ES+) m/z 149 [M+H]⁺.

### 2-(Methylthio)-4-pyrimidinecarbonitrile

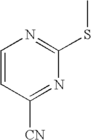

### Step 1. 4-Iodo-2-(methylthio)pyrimidine

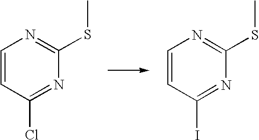

4-Chloro-2-(methylthio)pyrimidine (10 g, 62 mmol) was dissolved in hydroiodic acid (47%, 70 mL), and the mixture was stirred at room temperature for 20 hours. The precipitate was filtered, washed with ether to give a yellow solid (20.14 g). The yellow solid was suspended in water (75 mL), and ammonium hydroxide (30%) was added to the suspension to adjust the pH to 10. The mixture was extracted with ether (2×100 mL), the extracts were combined, washed with brine, dried (Na₂SO₄), filtered and concentrated to afford the title compound (14.14 g, 90%) as a yellow solid. MS (ES+) m/z 253 [M+H]⁺.

### Step 2. 2-(Methylthio)-4-pyrimidinecarbonitrile

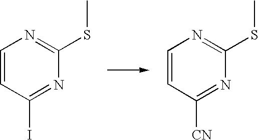

A mixture of compound from step 1 (7 g, 27.5 mmol) and copper cyanide (2.46 g, 27.5 mmol) in pyridine (150 mL) was heated at reflux for 4 hours. The reaction mixture was cooled to room temperature, concentrated, filtered, and washed with ether. The filtrate was washed with water, brine, dried (Na₂SO₄), filtered, and concentrated. The residue was purified on silica gel using 10% ethyl acetate-hexane to afford the title compound (3.2 g, 77%) as a white solid. MS (ES+) m/z 152 [M+H]⁺.

### 3-Chloro-6-fluoro-1H-indazol-5-amine

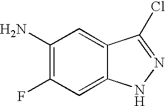

### Step 1. 3-Chloro-6-fluoro-5-nitro-1H-indazole

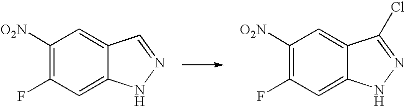

6-Fluoro-5-nitroindazole (850 mg, 4.693 mmol, 1.0 equiv) was dissolved in 20 mL EtOH, NaOCl (10 mL, 164.26 mmol, 35 equiv) was added and the reaction mixture was stirred at room temperature for 3 hours. The reaction mixture was concentrated en vacuo to remove EtOH was then diluted with EtOAc and water. The phases were separated, and the organic phase was washed once with satd. NaHCO₃, and once with satd. NaCl. The organic phase was dried over Na₂SO₄, filtered, and concentrated in vacuo. The residue was purified by flash chromatography (10-100% EtOAc in Hexanes) to provide 583 mg (58%) of the title compound as a light orange solid. MS (ES+) m/e 216 [M+H]⁺

### Step 2. 3-Chloro-6-fluoro-1H-indazol-5-amine

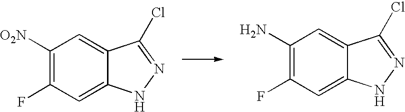

The product from Step 1 (575 mg, 2.667 mmol, 1.0 equiv) was dissolved in 15 mL EtOH and SnCl₂ (2.5 g, 13.337 mmol, 5.0 equiv) was added. The reaction mixture was heated to 70° C. for 4 hours. The reaction mixture was diluted with EtOAc and 6N NaOH, filtered through a pad of celite and washed with EtOAc and water. The phases of the filtrate were separated, and the organic phase was washed once with satd. NaHCO₃, and once with satd. NaCl. The organic phase was dried over Na₂SO₄, filtered, and concentrated en vacuo. The residue was purified by flash chromatography (20-100% EtOAc in Hexanes) to provide 250 mg (51%) of the title compound as a yellow solid. MS (ES+) m/e 186 [M+H]⁺

### ROCK Kinase Assay:

ROCK inhibitor activity was determined using human recombinant ROCK1 kinase domain (amino acid 2-543) expressed in Sf9 cells (see WO9967283). The enzyme was purified using His-tag NTA column and Source15 HPLC chromatography. The assay of Rock-1 activity involved incubation with peptide substrate and ATP³³, the subsequent incorporation of P³³ into the peptide was quantified by Scintillation Proximity Assay (SPA—Amersham Pharmacia).

For IC₅₀ determination, test compounds were typically dissolved at 10 mM in 100% DMSO, with subsequent serial dilution in 100% DMSO. Compounds were typically assayed over an eleven point dilution range with a concentration in the assay of 50 uM to 0.8 nM, in 3-fold dilutions. IC50 values were calculated by bespoke curve fitting software and then converted to pIC50. In some cases, compounds were assayed over an eleven point dilution range with a concentration in the assay of 10 uM to 0.8 nM, in 3-fold dilutions. For dose response curves in these cases, data were normalized and expressed as % inhibition using the formula 100*((U−C1)/(C2−C1)) where U is the unknown value, C1 is the average of the high signal (0%) control wells, and C2 is the average of the low signal (100%) control wells. Curve fitting was performed with the following equation: y=A+((B−A)/(1+(10̂x/10̂C)̂D)), where A is the minimum response, B is the maximum response, C is the log 10IC50, and D is the Hill slope. The results for each compound were recorded as pIC50 values (—C in the above equation). All Examples analyzed gave demonstrable activity in the ROCK kinase assay except compound Example number 98 which was not active in the range tested (10 uM to 0.8 nM).

Assays were performed in opaque, white walled, 384 well plates, in a total assay volume of 20 ul. In some cases, the total assay volume was 10 ul. The assays contained: 1 nM hROCK1; 1 uM biotinylated peptide (biotin-Ahx-AKRRRLSSLRA-CONH2); 1 uM ATP; 1.85 kBq per well ATP(γ-33P); 25 mM Hepes pH 7.4; 15 mM MgCl₂; 0.015% BSA. The reactions were incubated at 22° C. for 120 minutes, then terminated by the addition of a 50 ul solution containing 60 mM EDTA and streptavidin PVT SPA beads. The SPA beads were added to a concentration of 0.14 mg per well. In some cases, the reactions were incubated at 22° C. for 90 minutes, then terminated by the addition of a 50 ul solution containing 60 mM EDTA and streptavidin PVT SPA beads at a concentration of 0.08 mg per well. Alternatively in cases where the total assay volume was 10 ul, the reactions were incubated at 22° C. for 90 minutes, then terminated by the addition of a 10 ul solution containing 150 mM EDTA and streptavidin coupled PS imaging beads at a concentration of 0.03 mg per well. In all cases, the plates were allowed to incubate at 22° C. for 10 minutes before centrifugation at 1500 rpm for 1 minute. P³³ incorporation was quantified by scintillation counting in a Packard TopCount. When the reaction was terminated by addition of a 10 ul solution, P33 incorporation was quantified by luminescence detection using a Perkin-Elmer Viewlux ultraHTS microplate imager.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

1. A compound according to formula (I), wherein: 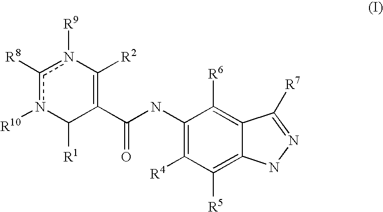
R⁸ is selected from the group consisting of aryl (optionally substituted with from 1-3 substituents selected from the group consisting of C₁₋₆ alkoxy, aryloxy, NR^{a}R^{b}, C₁₋₆ alkyl, SO₂C₁₋₆ alkyl, hydroxyl, halogen, cyano, nitro, C₁₋₆ thioalkyl, COOH, CO₂C₁₋₆ alkyl, and CONR^{a}R^{b}); NR^{a}R^{b}; heteroaryl (optionally substituted with 1-3 substituents selected from the group consisting of C₁₋₆ alkyl, NR^{a}R^{b}, halogen, cyano, nitro, C₁₋₆ alkoxy, aryloxy, COOH, CO₂C₁₋₆ alkyl, and CONR^{a}R^{b}, and C₁₋₆ thioalkyl); C₁₋₆ alkyl; C₁₋₆ alkylaryloxy; C₁₋₆ alkylaryl and C₃₋₇cycloalkyl;
R⁹ is absent; hydrogen; or C₁₋₃alkyl;
R¹⁰ is absent; hydrogen; or C₁₋₃alkyl;
R¹ is selected from the group consisting of aryl (optionally substituted with 1-3 substituents selected from the group consisting of halogen, cyano, nitro, C₁₋₆alkyl, SO₂C₁₋₆alkyl, and SO₂NR^{c}R^{d}); C₁₋₆alkylaryl (wherein said alkyl portion is optionally substituted with thiophenyl); acylaryl; heteroaryl (optionally substituted with 1-3 substituents selected from the group consisting of halogen, C₁₋₃alkyl and aryl); C₁₋₆ alkyl; and C₃₋₇ cycloalkyl;
R² is hydrogen or C₁₋₃ alkyl;
R⁶ is hydrogen;
R⁴ is hydrogen; C₁₋₃ alkyl; or halogen;
R⁵ is hydrogen; C₁₋₃ alkyl; or halogen;
R⁷ is hydrogen; halogen; or C₁₋₃ alkyl; and
R^{a} and R^{b} are each independently selected from the group consisting of hydrogen; C₁₋₆ alkyl; aryl; or taken together represent a ring containing from between 5 to 7 backbone atoms, wherein said ring is optionally substituted with from 1-3 substituents selected from C₁₋₃ alkyl; halogen; amino; or C₁₋₃ alkoxy; and one of the ring atoms optionally may be an additional heteroatom wherein said heteroatom is selected from the group consisting of N, O, or S);
R^{c} and R^{d} are each independently selected from the group consisting of hydrogen and C₁₋₃alkyl; and
pharmaceutically acceptable salt or solvate thereof.

2. A compound according to claim (I), wherein:
R⁸ is optionally substituted pyridyl.

3. A compound according to claim 2, wherein:
R⁸ is optionally substituted 4-pyridyl.

4. A compound according to claim 1, wherein:
R⁸ is optionally substituted phenyl.

5. A compound according to claim 4, wherein:
R⁸ contains a 4-substituted phenyl.

6. A compound according to claim 1, wherein:
R⁴ is hydrogen or fluorine.

7. A compound according to claim 1, wherein:
R⁵ is hydrogen or fluorine.

8. A compound according to claim 1, wherein:
R⁷ is hydrogen, methyl or chlorine.

9. A compound according to claim 1, wherein:
R¹ is substituted phenyl.

10. A compound according to claim 1, wherein:
R⁹ and R¹⁰ are each independently hydrogen or absent.

11. A compound according to claim 1, wherein the compound is:
a) 4-(4-chloro-2-fluorophenyl)-N-1H-indazol-5-yl-6-methyl-2-[2-(methyloxy)-4-pyridinyl]-1,4-dihydro-5-pyrimidinecarboxamide;
b) 2-amino-4-(4-fluorophenyl)-N-1H-indazol-5-yl-6-methyl-1,4-dihydro-5-pyrimidinecarboxamide;
c) 4-(4-fluorophenyl)-N-1H-indazol-5-yl-2,6-dimethyl-1,4-dihydro-5-pyrimidinecarboxamide;
d) 4-(4-fluorophenyl)-N-1H-indazol-5-yl-6-methyl-2-(phenylamino)-1,4-dihydro-5-pyrimidinecarboxamide;
e) 2-cyclopropyl-4-(4-fluorophenyl)-N-1H-indazol-5-yl-6-methyl-1,4-dihydro-5-pyrimidinecarboxamide;
f) 4-(4-fluorophenyl)-N-1H-indazol-5-yl-6-methyl-2-phenyl-1,4-dihydro-5-pyrimidinecarboxamide;
g) N-1H-indazol-5-yl-2-(4-methoxyphenyl)-6-methyl-4-[4-(trifluoromethyl)phenyl]-1,4-dihydropyrimidine-5-carboxamide;
h) 4-(4-fluorophenyl)-N-1H-indazol-5-yl-6-methyl-2-(3-methylphenyl)-1,4-dihydro-5-pyrimidinecarboxamide;
i) 4-(4-fluorophenyl)-N-1H-indazol-5-yl-6-methyl-2-[4-(trifluoromethyl)phenyl]-1,4-dihydro-5-pyrimidinecarboxamide;
j) 4-(4-fluorophenyl)-N-1H-indazol-5-yl-6-methyl-2-[3-(trifluoromethyl)phenyl]-1,4-dihydro-5-pyrimidinecarboxamide;
k) 2-(4-chlorophenyl)-4-(4-fluorophenyl)-N-1H-indazol-5-yl-6-methyl-1,4-dihydro-5-pyrimidinecarboxamide;
l) N-1H-indazol-5-yl-4-methyl-2-(3-methylphenyl)-6-[4-(trifluoromethyl)phenyl]-1,6-dihydro-5-pyrimidinecarboxamide;
m) N-1H-indazol-5-yl-4-methyl-2,6-bis[4-(trifluoromethyl)phenyl]-1,6-dihydro-5-pyrimidinecarboxamide;
n) N-1H-indazol-5-yl-4-methyl-2-[3-(trifluoromethyl)phenyl]-6-[4-(trifluoromethyl)phenyl]-1,6-dihydro-5-pyrimidinecarboxamide;
o) 2-(4-chlorophenyl)-N-1H-indazol-5-yl-4-methyl-6-[4-(trifluoromethyl)phenyl]-1,6-dihydro-5-pyrimidinecarboxamide;
p) 2-(3-fluoro-4-methylphenyl)-N-1H-indazol-5-yl-4-methyl-6-[4-(trifluoromethyl)phenyl]-1,6-dihydro-5-pyrimidinecarboxamide;
q) 4-(4-fluorophenyl)-N-1H-indazol-5-yl-6-methyl-2-(4-methylphenyl)-1,4-dihydro-5-pyrimidinecarboxamide;
r) 2-(3-fluoro-4-methylphenyl)-4-(4-fluorophenyl)-N-1H-indazol-5-yl-6-methyl-1,4-dihydro-5-pyrimidinecarboxamide;
s) N-1H-indazol-5-yl-4-methyl-2-(4-methylphenyl)-6-[4-(trifluoromethyl)phenyl]-1,6-dihydro-5-pyrimidinecarboxamide;
t) 2-(4-chlorophenyl)-N-1H-indazol-5-yl-4-methyl-6-(2-phenylethyl)-1,6-dihydro-5-pyrimidinecarboxamide;
u) 2-(3-fluoro-4-methylphenyl)-N-1H-indazol-5-yl-4-methyl-6-(2-phenylethyl)-1,6-dihydro-5-pyrimidinecarboxamide;
v) N-1H-indazol-5-yl-4-methyl-6-(2-phenylethyl)-2-[4-(trifluoromethyl)phenyl]-1,6-dihydro-5-pyrimidinecarboxamide;
w) N-1H-indazol-5-yl-4-methyl-6-(2-phenylethyl)-2-[3-(trifluoromethyl)phenyl]-1,6-dihydro-5-pyrimidinecarboxamide;
x) N-1H-indazol-5-yl-4-methyl-2-(3-methylphenyl)-6-(2-phenylethyl)-1,6-dihydro-5-pyrimidinecarboxamide;
y) N-1H-indazol-5-yl-4-methyl-2-(4-methylphenyl)-6-(2-phenylethyl)-1,6-dihydro-5-pyrimidinecarboxamide;
z) 2-[4-(aminocarbonyl)phenyl]-N-1H-indazol-5-yl-4-methyl-6-(2-phenylethyl)-1,6-dihydro-5-pyrimidinecarboxamide;
aa) 4-(4-fluorophenyl)-N-1H-indazol-5-yl-6-methyl-2-(2-pyridinyl)-1,4-dihydro-5-pyrimidinecarboxamide;
ab) 4-(4-fluorophenyl)-N-1H-indazol-5-yl-6-methyl-2-[6-(trifluoromethyl)-3-pyridinyl]-1,4-dihydro-5-pyrimidinecarboxamide;
ac) 4-(4-fluorophenyl)-N-1H-indazol-5-yl-6-methyl-2-(2-pyrazinyl)-1,4-dihydro-5-pyrimidinecarboxamide;
ad) 4-(4-fluorophenyl)-N-1H-indazol-5-yl-6-methyl-2-(2-thienyl)-1,4-dihydro-5-pyrimidinecarboxamide;
ae) 4-(4-fluorophenyl)-N-1H-indazol-5-yl-6-methyl-2-(3-pyridinyl)-1,4-dihydro-5-pyrimidinecarboxamide;
af) 4-(4-fluorophenyl)-N-1H-indazol-5-yl-6-methyl-2-(2-methyl-1,3-thiazol-4-yl)-1,4-dihydro-5-pyrimidinecarboxamide;
ag) 2-(2,3-dihydro-1,4-benzodioxiN-2-yl)-4-(4-fluorophenyl)-N-1H-indazol-5-yl-6-methyl-1,4-dihydro-5-pyrimidinecarboxamide;
ah) 4-(4-fluorophenyl)-N-1H-indazol-5-yl-6-methyl-2-[(phenyloxy)methyl]-1,4-dihydro-5-pyrimidinecarboxamide;
ai) 4-(4-fluorophenyl)-N-1H-indazol-5-yl-6-methyl-2-(4-pyridinyl)-1,4-dihydro-5-pyrimidinecarboxamide;
aj) 4-(4-fluorophenyl)-N-1H-indazol-5-yl-6-methyl-2-(1-oxido-2-pyridinyl)-1,4-dihydro-5-pyrimidinecarboxamide;
ak) 2-(4-chlorophenyl)-N-1H-indazol-5-yl-4-methyl-6-(phenylcarbonyl)-1,6-dihydro-5-pyrimidinecarboxamide;
al) N-1H-indazol-5-yl-4-methyl-6-phenyl-2-[4-(trifluoromethyl)phenyl]-1,6-dihydro-5-pyrimidinecarboxamide;
am) N-1H-indazol-5-yl-4-methyl-2-[3-(methyloxy)phenyl]-6-[4-(trifluoromethyl)phenyl]-1,6-dihydro-5-pyrimidinecarboxamide;
an) 4-[2-fluoro-4-(trifluoromethyl)phenyl]-N-1H-indazol-5-yl-6-methyl-2-[4-(trifluoromethyl)phenyl]-1,4-dihydro-5-pyrimidinecarboxamide;
ao) 4-(4-chloro-2-fluorophenyl)-N-1H-indazol-5-yl-6-methyl-2-[4-(trifluoromethyl)phenyl]-1,4-dihydro-5-pyrimidinecarboxamide;
ap) N-1H-indazol-5-yl-4-methyl-6-(5-phenyl-2-thienyl)-2-[4-(trifluoromethyl)phenyl]-1,6-dihydro-5-pyrimidinecarboxamide;
aq) N-1H-indazol-5-yl-4-methyl-2-(4-pyridinyl)-6-[4-(trifluoromethyl)phenyl]-1,6-dihydro-5-pyrimidinecarboxamide;
ar) N-1H-indazol-5-yl-4-methyl-6-(5-phenyl-2-thienyl)-2-(4-pyridinyl)-1,6-dihydro-5-pyrimidinecarboxamide;
as) N-1H-indazol-5-yl-4,6-dimethyl-2-[4-(methyloxy)phenyl]-1,4-dihydro-5-pyrimidinecarboxamide;
at) 4-(4-chloro-2-fluorophenyl)-N-1H-indazol-5-yl-6-methyl-2-[4-(methyloxy)phenyl]-1,4-dihydro-5-pyrimidinecarboxamide;
au) 4-[2-fluoro-4-(trifluoromethyl)phenyl]-N-1H-indazol-5-yl-6-methyl-2-[4-(methyloxy)phenyl]-1,4-dihydro-5-pyrimidinecarboxamide;
av) N-1H-indazol-5-yl-4-methyl-2-[4-(methyloxy)phenyl]-6-(5-phenyl-2-thienyl)-1,6-dihydro-5-pyrimidinecarboxamide;
aw) 4-(1-benzofuraN-2-yl)-N-1H-indazol-5-yl-6-methyl-2-[4-(methyloxy)phenyl]-1,4-dihydro-5-pyrimidinecarboxamide;
ax) N-1H-indazol-5-yl-4-methyl-6-[2-phenyl-2-(phenylthio)ethyl]-2-[4-(trifluoromethyl)phenyl]-1,6-dihydro-5-pyrimidinecarboxamide;
ay) 4-(1,3-benzodioxol-5-yl)-N-1H-indazol-5-yl-6-methyl-2-(4-pyridinyl)-1,4-dihydro-5-pyrimidinecarboxamide;
az) 4-[2-fluoro-4-(trifluoromethyl)phenyl]-N-1H-indazol-5-yl-6-methyl-2-(4-pyridinyl)-1,4-dihydro-5-pyrimidinecarboxamide;
ba) 4-(4-chloro-2-fluorophenyl)-N-1H-indazol-5-yl-6-methyl-2-(4-pyridinyl)-1,4-dihydro-5-pyrimidinecarboxamide;
bb) N-1H-indazol-5-yl-4-methyl-2-[4-(methylthio)phenyl]-6-[4-(trifluoromethyl)phenyl]-1,6-dihydro-5-pyrimidinecarboxamide;
bc) 2-(2,3-dimethylphenyl)-N-1H-indazol-5-yl-4-methyl-6-[4-(trifluoromethyl)phenyl]-1,6-dihydro-5-pyrimidinecarboxamide;
bd) 2-(2,5-dimethylphenyl)-N-1H-indazol-5-yl-4-methyl-6-[4-(trifluoromethyl)phenyl]-1,6-dihydro-5-pyrimidinecarboxamide;
be) 2-(4-chloro-3-methylphenyl)-N-1H-indazol-5-yl-4-methyl-6-[4-(trifluoromethyl)phenyl]-1,6-dihydro-5-pyrimidinecarboxamide;
bf) 2-[2-chloro-5-(trifluoromethyl)phenyl]-N-1H-indazol-5-yl-4-methyl-6-[4-(trifluoromethyl)phenyl]-1,6-dihydro-5-pyrimidinecarboxamide;
bg) 2-[3,4-bis(methyloxy)phenyl]-N-1H-indazol-5-yl-4-methyl-6-[4-(trifluoromethyl)phenyl]-1,6-dihydro-5-pyrimidinecarboxamide;
bh) N-1H-indazol-5-yl-4-methyl-2-[2-(methyloxy)phenyl]-6-[4-(trifluoromethyl)phenyl]-1,6-dihydro-5-pyrimidinecarboxamide;
bi) 2-[2,3-bis(methyloxy)phenyl]-N-1H-indazol-5-yl-4-methyl-6-[4-(trifluoromethyl)phenyl]-1,6-dihydro-5-pyrimidinecarboxamide;
bj) 2-(2-chlorophenyl)-N-1H-indazol-5-yl-4-methyl-6-[4-(trifluoromethyl)phenyl]-1,6-dihydro-5-pyrimidinecarboxamide;
bk) 2-(3-chlorophenyl)-N-1H-indazol-5-yl-4-methyl-6-[4-(trifluoromethyl)phenyl]-1,6-dihydro-5-pyrimidinecarboxamide;
bl) 4-(4-chloro-2-fluorophenyl)-2-(2-chloro-4-pyridinyl)-N-1H-indazol-5-yl-6-methyl-1,4-dihydro-5-pyrimidinecarboxamide;
bm) 2-(2-chloro-4-pyridinyl)-N-1H-indazol-5-yl-4-methyl-6-[4-(trifluoromethyl)phenyl]-1,6-dihydro-5-pyrimidinecarboxamide;
bn) 2-(2-chloro-4-pyridinyl)-4-[2-fluoro-4-(trifluoromethyl)phenyl]-N-1H-indazol-5-yl-6-methyl-1,4-dihydro-5-pyrimidinecarboxamide;
bo) 4-(4-chloro-2-fluorophenyl)-N-1H-indazol-5-yl-6-methyl-2-(4-morpholinyl)-1,4-dihydro-5-pyrimidinecarboxamide;
bp) 4-(4-chloro-2-fluorophenyl)-N-1H-indazol-5-yl-6-methyl-2-{[4-(methyloxy)phenyl]methyl}-1,4-dihydro-5-pyrimidinecarboxamide;
bq) 4-(4-chloro-2-fluorophenyl)-N-1H-indazol-5-yl-6-methyl-2-(2-thioxo-2,3-dihydro-1H-benzimidazol-5-yl)-1,4-dihydro-5-pyrimidinecarboxamide;
br) 2-(2-chloro-4-pyridinyl)-N-1H-indazol-5-yl-4,6-dimethyl-1,4-dihydro-5-pyrimidinecarboxamide;
bs) 4-(4-chloro-2-fluorophenyl)-2-(2-chloro-4-pyridinyl)-N-(6-fluoro-1H-indazol-5-yl)-6-methyl-1,4-dihydro-5-pyrimidinecarboxamide;
bt) 4-(4-chloro-2-fluorophenyl)-N-1H-indazol-5-yl-6-methyl-2-[6-(methyloxy)-3-pyridinyl]-1,4-dihydro-5-pyrimidinecarboxamide;
bu) 4-(4-chloro-2-fluorophenyl)-2-(2,6-dichloro-4-pyridinyl)-N-1H-indazol-5-yl-6-methyl-1,4-dihydro-5-pyrimidinecarboxamide;
by) N-1H-indazol-5-yl-4-methyl-2-[4-(methyloxy)phenyl]-6-phenyl-1,6-dihydro-5-pyrimidinecarboxamide;
bw) 4-(4-chloro-2-fluorophenyl)-2-{2-[(4-chlorophenyl)oxy]-4-pyridinyl}-N-1H-indazol-5-yl-6-methyl-1,4-dihydro-5-pyrimidinecarboxamide;
by) 4-[4-(aminosulfonyl)phenyl]-N-1H-indazol-5-yl-6-methyl-2-[4-(methyloxy)phenyl]-1,4-dihydro-5-pyrimidinecarboxamide;
bz) N-1H-indazol-5-yl-4-methyl-2-[4-(methyloxy)phenyl]-6-[4-(methylsulfonyl)phenyl]-1,6-dihydro-5-pyrimidinecarboxamide;
ca) 4-(4-chloro-2-fluorophenyl)-2-[(4-chlorophenyl)methyl]-N-1H-indazol-5-yl-6-methyl-1,4-dihydro-5-pyrimidinecarboxamide;
cb) 4-(4-chloro-2-fluorophenyl)-N-1H-indazol-5-yl-6-methyl-2-(methylsulfonyl)-1,4-dihydro-5-pyrimidinecarboxamide;
cc) 2-(2-chloro-4-pyridinyl)-4-(4-fluorophenyl)-N-1H-indazol-5-yl-6-methyl-1,4-dihydro-5-pyrimidinecarboxamide;
cd) 4-(1,3-benzodioxol-5-yl)-2-(2-chloro-4-pyridinyl)-N-1H-indazol-5-yl-6-methyl-1,4-dihydro-5-pyrimidinecarboxamide;
ce) 2-(2-chloro-4-pyridinyl)-N-1H-indazol-5-yl-4-methyl-6-(2-methylpropyl)-1,6-dihydro-5-pyrimidinecarboxamide;
cf) 2-(2-chloro-4-pyridinyl)-4-(2,2-dimethylpropyl)-N-1H-indazol-5-yl-6-methyl-1,4-dihydro-5-pyrimidinecarboxamide;
cg) 2-(2-chloro-4-pyridinyl)-4-cyclopropyl-N-1H-indazol-5-yl-6-methyl-1,4-dihydro-5-pyrimidinecarboxamide;
ch) 2-(2-chloro-4-pyridinyl)-N-1H-indazol-5-yl-4-methyl-6-(phenylmethyl)-1,6-dihydro-5-pyrimidinecarboxamide;
ci) 2-(2-chloro-4-pyridinyl)-N-1H-indazol-5-yl-4-methyl-6-(2-phenylethyl)-1,6-dihydro-5-pyrimidinecarboxamide;
cj) 2-(2-chloro-4-pyridinyl)-4-ethyl-N-1H-indazol-5-yl-6-methyl-1,4-dihydro-5-pyrimidinecarboxamide;
ck) 2-(2-chloro-4-pyridinyl)-N-1H-indazol-5-yl-4-methyl-6-propyl-1,6-dihydro-5-pyrimidinecarboxamide;
cl) 2-(2-chloro-4-pyridinyl)-4-(1,1-dimethylethyl)-N-1H-indazol-5-yl-6-methyl-1,4-dihydro-5-pyrimidinecarboxamide;
cm) 4-(1,3-benzodioxol-5-yl)-N-(6-fluoro-1H-indazol-5-yl)-6-methyl-2-[2-(methyloxy)-4-pyridinyl]-1,4-dihydro-5-pyrimidinecarboxamide;
cn) N-(6-fluoro-1H-indazol-5-yl)-4-methyl-2-[2-(methyloxy)-4-pyridinyl]-6-[4-(trifluoromethyl)phenyl]-1,6-dihydro-5-pyrimidinecarboxamide;
co) N-(6-fluoro-1H-indazol-5-yl)-4-[2-fluoro-4-(trifluoromethyl)phenyl]-6-methyl-2-[2-(methyloxy)-4-pyridinyl]-1,4-dihydro-5-pyrimidinecarboxamide;
cp) 4-(4-chloro-2-fluorophenyl)-N-(6-fluoro-1H-indazol-5-yl)-6-methyl-2-[2-(methyloxy)-4-pyridinyl]-1,4-dihydro-5-pyrimidinecarboxamide;
cq) N-(6-fluoro-1H-indazol-5-yl)-4-(4-fluorophenyl)-6-methyl-2-[2-(methyloxy)-4-pyridinyl]-1,4-dihydro-5-pyrimidinecarboxamide;
cr) N-(6-fluoro-1H-indazol-5-yl)-4-(4-fluorophenyl)-6-methyl-2-[6-(methyloxy)-3-pyridinyl]-1,4-dihydro-5-pyrimidinecarboxamide;
cs) 4-(4-chloro-2-fluorophenyl)-2-(2-chloro-4-pyridinyl)-N-(6-fluoro-1H-indazol-5-yl)-1,6-dimethyl-1,4-dihydro-5-pyrimidinecarboxamide;
ct) 4-(1,3-benzodioxol-5-yl)-N-(6-fluoro-1H-indazol-5-yl)-6-methyl-2-[6-(methyloxy)-3-pyridinyl]-1,4-dihydro-5-pyrimidinecarboxamide;
cu) N-(6-fluoro-1H-indazol-5-yl)-4-methyl-2-[6-(methyloxy)-3-pyridinyl]-6-[4-(trifluoromethyl)phenyl]-1,6-dihydro-5-pyrimidinecarboxamide;
cv) N-(6-fluoro-1H-indazol-5-yl)-4-[2-fluoro-4-(trifluoromethyl)phenyl]-6-methyl-2-[6-(methyloxy)-3-pyridinyl]-1,4-dihydro-5-pyrimidinecarboxamide;
cw) 4-(4-chloro-2-fluorophenyl)-N-(6-fluoro-1H-indazol-5-yl)-6-methyl-2-[6-(methyloxy)-3-pyridinyl]-1,4-dihydro-5-pyrimidinecarboxamide;
cz) 4-(4-chlorophenyl)-2-(2-chloro-4-pyridinyl)-N-(6-fluoro-1H-indazol-5-yl)-6-methyl-1,4-dihydro-5-pyrimidinecarboxamide;
da) 4-(4-chlorophenyl)-N-(6-fluoro-1H-indazol-5-yl)-6-methyl-2-[2-(methyloxy)-4-pyridinyl]-1,4-dihydro-5-pyrimidinecarboxamide;
db) 4-(4-chlorophenyl)-N-(6-fluoro-1H-indazol-5-yl)-6-methyl-2-[6-(methyloxy)-3-pyridinyl]-1,4-dihydro-5-pyrimidinecarboxamide;
dc) N-(3-chloro-6-fluoro-1H-indazol-5-yl)-4-(4-chloro-2-fluorophenyl)-2-(2-chloro-4-pyridinyl)-6-methyl-1,4-dihydro-5-pyrimidinecarboxamide;
dd) N-(3-chloro-6-fluoro-1H-indazol-5-yl)-4-(4-chloro-2-fluorophenyl)-6-methyl-2-[2-(methyloxy)-4-pyridinyl]-1,4-dihydro-5-pyrimidinecarboxamide;
de) 4-(4-chloro-2-fluorophenyl)-N-1H-indazol-5-yl-2-[4-(methyloxy)phenyl]-6-(trifluoromethyl)-1,4-dihydro-5-pyrimidinecarboxamide;
df) 2-amino-4-(4-chlorophenyl)-N-(6-fluoro-1H-indazol-5-yl)-6-methyl-1,4-dihydro-5-pyrimidinecarboxamide;
dg) 4-(4-chlorophenyl)-N-(6-fluoro-1H-indazol-5-yl)-2,6-dimethyl-1,4-dihydro-5-pyrimidinecarboxamide;
dh) 4-(4-chlorophenyl)-2-[2-(ethyloxy)-4-pyridinyl]-N-(6-fluoro-1H-indazol-5-yl)-6-methyl-1,4-dihydro-5-pyrimidinecarboxamide;
di) 2-[2-(ethyloxy)-4-pyridinyl]-N-(6-fluoro-1H-indazol-5-yl)-4-[2-fluoro-4-(trifluoromethyl)phenyl]-6-methyl-1,4-dihydro-5-pyrimidinecarboxamide;
dj) 4-(4-chloro-2-fluorophenyl)-2-[2-(ethyloxy)-4-pyridinyl]-N-(6-fluoro-1H-indazol-5-yl)-6-methyl-1,4-dihydro-5-pyrimidinecarboxamide;
dk) 4-(4-chlorophenyl)-N-(6-fluoro-1H-indazol-5-yl)-6-methyl-2-(2-methyl-4-pyridinyl)-1,4-dihydro-5-pyrimidinecarboxamide;
dl) N-(6-fluoro-1H-indazol-5-yl)-4-[2-fluoro-4-(trifluoromethyl)phenyl]-6-methyl-2-(2-methyl-4-pyridinyl)-1,4-dihydro-5-pyrimidinecarboxamide;
dm) 4-(4-chloro-2-fluorophenyl)-N-(6-fluoro-1H-indazol-5-yl)-6-methyl-2-(2-methyl-4-pyridinyl)-1,4-dihydro-5-pyrimidinecarboxamide;
dn) 4-(4-chlorophenyl)-N-(6-fluoro-1H-indazol-5-yl)-6-methyl-2′-(methylthio)-1,4-dihydro-2,4′-bipyrimidine-5-carboxamide;
do) 4-(4-chlorophenyl)-2-(2-chloro-4-pyridinyl)-6-methyl-N-(3-methyl-1H-indazol-5-yl)-1,4-dihydro-5-pyrimidinecarboxamide;
dp) 4-(4-chloro-2-fluorophenyl)-2-(2-chloro-4-pyridinyl)-6-methyl-N-(3-methyl-1H-indazol-5-yl)-1,4-dihydro-5-pyrimidinecarboxamide;
dq) 2-(2-chloro-4-pyridinyl)-4-[2-fluoro-4-(trifluoromethyl)phenyl]-6-methyl-N-(3-methyl-1H-indazol-5-yl)-1,4-dihydro-5-pyrimidinecarboxamide;
dr) N-(3-chloro-1H-indazol-5-yl)-4-(4-chlorophenyl)-2-(2-chloro-4-pyridinyl)-6-methyl-1,4-dihydro-5-pyrimidinecarboxamide;
ds) 4-(4-chloro-2-fluorophenyl)-N-(3-chloro-1H-indazol-5-yl)-2-(2-chloro-4-pyridinyl)-6-methyl-1,4-dihydro-5-pyrimidinecarboxamide;
dt) 4-(4-Fluorophenyl)-N-1H-indazol-5-yl-6-methyl-2-[4-(methyloxy)phenyl]-1,4-dihydro-5-pyrimidinecarboxamide;
du) 6-(4-Chloro-2-fluorophenyl)-N-1H-indazol-5-yl-1,4-dimethyl-2-[4-(methyloxy)phenyl]-1,6-dihydro-5-pyrimidinecarboxamide; or a pharmaceutically acceptable salt or solvate thereof.

12. A composition comprising a compound according to claim 1 and a pharmaceutically acceptable excipient.

13. A method of treating a disease associated with excess Rho-kinase activity comprising the administration of a compound of claim 1 to a mammal in need thereof.

14. A method of treating hypertension, alzheimer's disease, chronic and congestive heart failure and ischemic angina comprising the administration of a compound of claim 1 to a mammal in need thereof.

15. The method of claim 14 wherein the mammal is human.

16. A process for the preparation of a compound of formula (I) of claim 1 comprising:
a) the coupling of a compound of formula (II) with an indazole of formula (III) 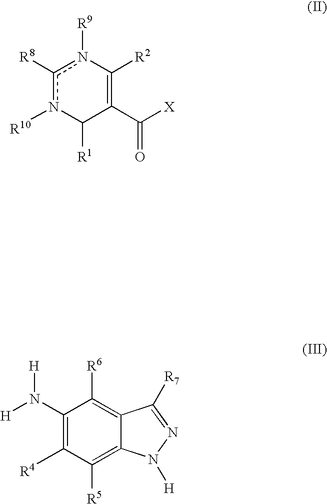
wherein R¹, R², R⁴, R⁵, R⁶, R⁷, R⁸, R⁹ and R¹⁰ are each as defined in formula (I) in claim 1, or are groups readily convertible thereto, and
x is OH, halogen, OC(═O)OC₁₋₆alkyl, or imidazole.

17. A process for the preparation of a compound of formula (I) of claim 1, comprising the reaction between compounds of formula (IV), (V) and (VI) in the presence of a base: 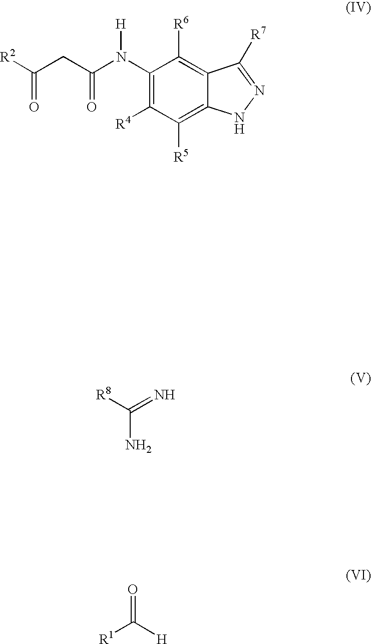
wherein R¹, R², R⁴, R⁵, R⁶, R⁷, and R⁸ are each as defined in formula (I) in claim 1, or are groups readily convertible thereto.
